(19) United States
(12) Patent Application Publication
(10) Pub. No.: US 2010/0261791 A1
(43) Pub. Date: Oct. 14, 2010

(54) Compositions and Methods for Improving or Preserving Brain Function

(75) Inventor: Samuel T. Henderson, Broomfield, CO (US)

Correspondence Address: SWANSON & BRATSCHUN, L.L.C., 8210 SOUTHPARK TERRACE, LITTLETON, CO, 80120, US

(73) Assignee: ACCERA, INC., Broomfield, CO (US)

(21) Appl. No.: 12/821,401

(22) Filed: Jun. 23, 2010

Related U.S. Application Data
(63) Continuation of application No. 11/611,114, filed on Dec. 14, 2006, now abandoned.
Application No. 11/611,114 is a continuation-in-part of application No. 11/021,920, filed on Dec. 22, 2004.
Application No. 11/021,920 is a continuation of application No. 10/152,147, filed on May 20, 2002, now Pat. No. 6,835,750.
Application No. 10/152,147 is a continuation-in-part of application No. 09/845,741, filed on May 1, 2001, now abandoned.
(60) Provisional application No. 60/200,980, filed on May 1, 2000.
(60) Provisional application No. 60/744,140, filed on Apr. 3, 2006.
(60) Provisional application No. 60/751,391, filed on Dec. 15, 2005.

(51) Int. Cl. A61K 31/23; C07C 69/527; A61K 31/22; C07C 69/347; A61P 25/28; A61P 25/00

(52) U.S. Cl. 514/547; 554/122; 560/224

(57) ABSTRACT

The present invention is related to mammalian nutrition and effects thereof in individuals with age associated cognitive decline such as Age Associated Memory Impairment (AAMI) or a dementing illness such as Alzheimer's disease or related dementia, or Mild Cognitive Impairment, such as improving performance in, or reversal, prevention, reducing and delaying decline in, one or more of cognitive function, memory, behavior, cerebrovascular function, motor function, and/or brain physiology are seen. In particular, the present invention utilizes medium chain triglycerides, in one embodiment, administered as part of a long-term treatment regimen, to preserve or improve learning, attention, motor performance, cerebrovascular function, social behavior, and to increase activity levels, particularly in aging mammals.

### RELATED APPLICATIONS

This application is a continuation of U.S. application Ser. No. 11/611,114, filed Dec. 14, 2006, currently pending, entitled, “Compositions and Methods for Improving or Preserving Brain Function,” which is a continuation-in-part of U.S. application Ser. No. 11/021,920, filed Dec. 22, 2004, currently pending, entitled “Use of Medium Chain Triglycerides for the Treatment and Prevention of Alzheimer's Disease and Other Diseases Resulting from Reduced Neuronal Metabolism II”, which is a continuation of U.S. application Ser. No. 10/152,147, filed May 20, 2002, now U.S. Pat. No. 6,835,750, issued Dec. 28, 2004, entitled “Use of Medium Chain Triglycerides for the Treatment and Prevention of Alzheimer's Disease and Other Diseases Resulting from Reduced Neuronal Metabolism II”, which is a continuation-in-part of U.S. application Ser. No. 09/845,741, filed May 1, 2001, now abandoned, entitled “Use of Medium Chain Trigylcerides for the Treatment and Prevention of Alzheimer's Disease and Other Diseases Resulting from Reduced Neuronal Metabolism,” which claims priority to U.S. Provisional Application Ser. No. 60/200,980 filed May 1, 2000, expired, entitled “Use of Medium Chain Triglycerides for the Treatment and Prevention of Alzheimer's Disease and Other Diseases Resulting from Reduced Neuronal Metabolism.” The U.S. application Ser. No. 11/611,114 application also claims the benefit of U.S. Provisional Application Ser. No. 60/774,140, filed Apr. 3, 2006, pending, entitled “Use of Medium Chain Triglycerides for Treatment and Prevention of Age-Associated Memory Impairment” and to U.S. Provisional Application Ser. No. 60/751,391, filed Dec. 15, 2005, pending, entitled “Compositions and Methods for Preserving Brain Function.”

### FIELD OF THE INVENTION

The present invention is related to mammalian nutrition and effects thereof in individuals with age associated cognitive decline such as Age Associated Memory Impairment (AAMI), or a dementing illness such as Alzheimer's disease, Mild Cognitive Impairment or other related dementia. Improvement in performance in, or reversal, prevention, reducing and delaying of decline in, any one of cognitive function, memory, behavior, cerebrovascular function, motor function, and/or brain physiology are seen. In particular, the present invention utilizes medium chain triglycerides, in one embodiment, administered as part of a treatment regimen, to preserve or improve learning, attention, motor performance, cerebrovascular function, social behavior, and to increase activity levels, particularly in aging mammals.

### BACKGROUND OF THE INVENTION

Aging causes deterioration of various aspects of physiology in normal adults, including memory performance. Impairment of memory performance in the elderly has been detected in several standard memory tests, including the Wechsler Memory Scale (WMS) and immediate and delayed Visual Reproduction Test, the Rey Auditory Verbal Learning Test (RAVLT) and others. The decline of memory performance with age has been referred to as Age Associated Memory Impairment (AAMI) (for review see (Larrabee, G. J. and Crook, T. H., 3rd, Estimated prevalence of age-associated memory impairment derived from standardized tests of memory function, Int Psychogeriatr, 1994, 6:95-104)). AAMI describes healthy people over 50 years of age who have experienced cognitive losses since early adult life that lie within the bounds of normality. Criteria for AAMI include both subjective and objective evidence that memory loss has occurred since early adult life in the absence of disease or trauma of possible etiologic significance. AAMI is distinct from Alzheimer's disease (AD). People with AAMI are not necessarily at greater risk for developing AD (Youngjohn, J. R. and Crook, T. H., 3rd, Learning, forgetting, and retrieval of everyday material across the adult life span, J Clin Exp Neuropsychol, 1993, 15:447-60) and are appropriately described as having “normal” age-related memory loss.

Cognitive impairment, progressive decline in cognitive function, changes in brain morphology, and changes in cerebrovascular function are commonly observed in aged or aging individuals. Age-related or age-associated cognitive impairment may manifest itself in many ways, and can include short-term memory loss, diminished capacity to learn or rate of learning, diminished attention, diminished motor performance, and/or dementia, among other indicia. In some cases, a specific etiology of such cognitive decline is unknown, while in other cases, cognitive impairment stems from the onset or progression of recognized diseases, disorders, or syndromes, for example, Alzheimer's Disease (AD). Age-associated cognitive decline is distinct from, and can occur independently of AD. One such specific recognized syndrome includes Age-Associated Memory Impairment (AAMI).

Alzheimer's Disease (AD) is a progressive neurodegenerative disorder, which primarily affects the elderly. There are two forms of AD, early-onset and late-onset. Early-onset AD is rare, strikes susceptible individuals as early as the third decade, and is frequently associated with mutations in a small set of genes. Late onset, or spontaneous AD, is common, strikes in the seventh or eighth decade, and is a multifactorial disease. Late-onset AD is the leading cause of dementia in persons over the age of 65. An estimated 7-10% of the American population over 65, and up to 40% of the American population greater than 80 years of age is afflicted with AD (McKhann et al., 1984; Evans et al. 1989). Early in the disease, patients experience loss of memory and orientation. As the disease progresses, additional cognitive functions become impaired, until the patient is completely incapacitated. Many theories have been proposed to describe the chain of events that give rise to AD, yet, at the time of this application, the cause remains unknown. Currently, no effective prevention or treatment exists for AD. The only drugs to treat AD on the market today include Aricept®, Cognex®, Reminyl® and Exelon® which are acetylcholinesterase inhibitors and Namenda™, an NMDA receptor antagonist. These drugs do not address the underlying pathology of AD. They merely enhance the effectiveness of those nerve cells still able to function and only provide symptomatic relief from the disease. Since the disease continues, the benefits of these treatments are slight.

Early-onset cases of AD are rare (−5%), occur before the age of 60 and are frequently associated with mutations in three genes, presenilin1 (PS1), presenilin2 (PS2) and amyloid precursor protein (APP) (for review see Selkoe, 1999). Early-onset AD cases exhibit cognitive decline and neuropathological lesions that are similar to those found in late-onset AD. AD is characterized by the accumulation of neurofibrillar tangles (NFT) and β-amyloid deposits in senile plaques (SP) and cerebral blood vessels. The main constituent of senile plaques is the β-amyloid peptide (Aβ), which is derived from the APP protein by proteolytic processing. The presenilin proteins may facilitate the cleavage of APP. The Aβ peptide is amyloidogenic and under certain conditions will form insoluble fibrils. However, the toxicity of Aβ peptide and fibrils remains controversial. In some cases Aβ has been shown to be neurotoxic, while others find it to be neurotrophic (for reviews see Selkoe, 1999). The cause of early-onset AD is hypothesized to be accumulation of aggregated proteins in susceptible neurons. Mutations in APP are hypothesized to lead to direct accumulation of fibrillar Aβ, while mutations in PS1 or PS2 are proposed to lead to indirect accumulation of Aβ. How a variety of mutations in PS1 and PS2 lead to increased Aβ accumulation has not been resolved. Accumulation of aggregated proteins is common to many progressive neurodegenerative disorders, including Amyloid Lateral Sclerosis (ALS) and Huntington's Disease (for review see Koo et al., 1999). Evidence suggests that accumulation of aggregated proteins inhibits cellular metabolism and ATP production. Consistent with this observation is the finding that buffering the energy capacity of neurons with creatine will delay the onset of ALS in transgenic mouse models (Klivenyi et al., 1999). Much of the prior art on AD has focused on inhibiting production of or aggregation of Aβ peptides; such as U.S. Pat. No. 5,817,626, U.S. Pat. No. 5,854,204, and U.S. Pat. No. 5,854,215. Other prior art to treat AD include, U.S. Pat. No. 5,385,915 entitled “Treatment of amyloidosis associated with Alzheimer Disease using modulators of protein phosphorylation” and U.S. Pat. No. 5,538,983 entitled “Method of treating amyloidosis by modulation of calcium.” Attempts to increase neuronal survival by use of nerve growth factors have dealt with either whole cell, gene or protein delivery, such as described in U.S. Pat. No. 5,650,148 entitled “Method of grafting genetically modified cells to treat defects, disease or damage of the central nervous system”, and U.S. Pat. No. 5,936,078 entitled “DNA and Protein for the Diagnosis and Treatment of Alzheimer's Disease.”

The vast majority (−95%) of AD cases are late-onset, occurring in the seventh or eighth decade. Late-onset AD is not associated with mutations in APP, PS1 or PS2, yet exhibits neuropathological lesions and symptoms that are similar to those found in early-onset AD. Since late-onset AD is the most common form, it will be referred to herein as AD, while early-onset AD will be referred to as such. The similar neuropathology and outward symptoms of early-onset and late-onset AD have led to the “amyloid cascade hypothesis of AD” (Selkoe, 1994). This model holds that both early- and late-onset AD result from accumulation of toxic amyloid deposits. The model speculates that in early-onset cases, amyloid accumulates rapidly, while in late-onset, amyloid accumulates slowly. Much of the research on prevention and treatment of AD has focused on inhibition of amyloid accumulation. However, the amyloid cascade hypothesis remains controversial. Amyloid deposits may be a marker for the disease and not the cause. Translation of Dr. Alzheimer's original work on the neuropathology of AD, relates that he did not favor the view that senile plaques were causative. He states “These changes are found in the basal ganglia, the medulla, the cerebellum and the spinal cord, although there are no plaques at all in those sites or only isolated ones. So we have to conclude that the plaques are not the cause of senile dementia but only an accompanying feature of senile involution of the central nervous system.” The italics are his own (Davis and Chisholm, 1999). Many years of research have not resolved this issue (for review of amyloid hypothesis, see Selkoe, 1999; for counter argument, see Neve et al., 1998). Since the present invention addresses the decreased neuronal metabolism associated with AD, it does not rely on the validity of the amyloid cascade hypothesis and represents a novel approach to treating AD and other similar diseases.

At the time of this application, the cause of AD remains unknown, yet a large body of evidence has made it clear that Alzheimer's disease is associated with decreased neuronal metabolism. In 1984, Blass and Zemcov proposed that AD results from a decreased metabolic rate in sub-populations of cholinergic neurons. However, it has become clear that AD is not restricted to cholinergic systems, but involves many types of transmitter systems, and several discrete brain regions. Positron-emission tomography has revealed poor glucose utilization in the brains of AD patients, and this disturbed metabolism can be detected well before clinical signs of dementia occur (Reiman, E. M., et al., Preclinical evidence of Alzheimer's disease in persons homozygous for the epsilon 4 allele for apolipoprotein E, N Engl J Med, 1996, 334:752-8); (Messier, C. and Gagnon, M., Glucose regulation and cognitive functions: relation to Alzheimer's disease and diabetes, Behav Brain Res, 1996, 75:1-11); (Frolich, L., et al., Brain insulin and insulin receptors in aging and sporadic Alzheimer's disease, J Neural Transm, 1998, 105:423-38). Additionally, certain populations of cells, such as somatostatin cells of the cortex in AD brain are smaller, and have reduced Golgi apparatus; both indicating decreased metabolic activity (for review see Swaab et al. 1998). Measurements of the cerebral metabolic rates in healthy versus AD patients demonstrated a 20-40% reduction in glucose metabolism in AD patients (Hoyer, 1992). Reduced glucose metabolism results in critically low levels of ATP in AD patients. Also, the severity of decreased metabolism was found to correlate with senile plaque density (Meier-Ruge, et al. 1994).

Additionally, molecular components of insulin signaling and glucose utilization are impaired in AD patients. Glucose is transported across the blood brain barrier and is used as a major fuel source in the adult brain. Consistent with the high level of glucose utilization, the brains of mammals are well supplied with receptors for insulin and IGF, especially in the areas of the cortex and hippocampus, which are important for learning and memory (Frolich, L., et al., Brain insulin and insulin receptors in aging and sporadic Alzheimer's disease, J Neural Transm, 1998, 105:423-38). In patients diagnosed with AD, increased densities of insulin receptor were observed in many brain regions, yet the level of tyrosine kinase activity that normally is associated with the insulin receptor was decreased, both relative to age-matched controls (Frolich, L., et al., Brain insulin and insulin receptors in aging and sporadic Alzheimer's disease, J Neural Transm, 1998, 105:423-38). The increased density of receptors represents up-regulation of receptor levels to compensate for decreased receptor activity. Activation of the insulin receptor is known to stimulate phosphatidylinositol-3 kinase (PI3K). PI3K activity is reduced in AD patients (Jolles et al., 1992; Zubenko et al., 1999). Furthermore, the density of the major glucose transporters in the brain, GLUT1 and GLUT3 were found to be 50% of age matched controls (Simpson and Davies, 1994). The disturbed glucose metabolism in AD has led to the suggestion that AD may be a form of insulin resistance in the brain, similar to type II diabetes (Hoyer, S., Risk factors for Alzheimer's disease during aging. Impacts of glucose/energy metabolism, J Neural Transm Suppl, 1998, 54:187-94). Inhibition of insulin receptor activity can be exogenously induced in the brains of rats by intracerebroventricular injection of streptozotocin, a known inhibitor of the insulin receptor. These animals develop progressive defects in learning and memory (Lannert, H. and Hoyer, S., Intracerebroventricular administration of streptozotocin causes long-term diminutions in learning and memory abilities and in cerebral energy metabolism in adult rats, Behav Neurosci, 1998, 112:1199-208). While glucose utilization is impaired in brains of AD patients, use of the ketone bodies, beta-hydroxybutyrate and acetoacetate is unaffected (Ogawa et al., 1996).

The cause of decreased neuronal metabolism in AD remains unknown. Yet, aging may exacerbate the decreased glucose metabolism in AD. Insulin stimulation of glucose uptake is impaired in the elderly, leading to decreased insulin action and increased insulin resistance (for review see Finch and Cohen, 1997). For example, after a glucose load, mean plasma glucose is 10-30% higher in those over 65 than in younger subjects. Hence, genetic risk factors for AD may result in slightly compromised neuronal metabolism in the brain. These defects would only become apparent later in life when glucose metabolism becomes impaired, and thereby contribute to the development of AD.

Attempts to compensate for reduced cerebral metabolic rates in AD patients have met with some success. Treatment of AD patients with high doses of glucose and insulin increases cognitive scores (Craft, S., et al., Memory improvement following induced hyperinsulinemia in Alzheimer's disease, Neurobiol Aging, 1996, 17:123-30). However, since insulin is a polypeptide and must be transported across the blood brain barrier, delivery to the brain is complicated. Therefore, insulin is administered systemically. A large dose of insulin in the blood stream can lead to hyperinsulinemia, which will cause irregularities in other tissues. Both of these shortcomings make this type of therapy difficult and rife with complications. Accordingly, there remains a need for an agent that may increase the cerebral metabolic rate and subsequently the cognitive abilities of a patient suffering from Alzheimer's disease.

Substantial scientific evidence has shown that declines in cerebral glucose metabolism occur during aging in several mammalian species, such as rhesus monkeys (Noda, A., et al., Age-related changes in cerebral blood flow and glucose metabolism in conscious rhesus monkeys, Brain Res, 2002, 936:76-81), dogs (London, E. D., et al., Regional cerebral metabolic rate for glucose in beagle dogs of different ages, Neurobiol Aging, 1983, 4:121-6) and humans (Moeller, J. R., et al., The metabolic topography of normal aging, J Cereb Blood Flow Metab, 1996, 16:385-98). The metabolic decreases occur in many regions of the brain and in some examples represent an approximately 12% decrease in global metabolic rate between the ages of 20 and 80 (Moeller, J. R., et al., The metabolic topography of normal aging, J Cereb Blood Flow Metab, 1996, 16:385-98). Such decreases in glucose metabolism may underlie cognitive decline seen with aging. Therefore improving metabolism in the brain may improve AAMI. This is consistent with studies in humans that have shown increases in cognition following raising serum glucose concentrations by oral glucose administration in the elderly groups but not for young groups (Hall, J. L., et al., Glucose enhancement of performance on memory tests in young and aged humans, Neuropsychologia, 1989, 27:1129-38). Such experiments have been replicated several times and seem to indicate that memory facilitation by glucose is characterized by an inverted-U shape, with too much glucose negating the effect (Parsons, M. W. and Gold, P. E., Glucose enhancement of memory in elderly humans: an inverted-U dose-response curve, Neurobiol Aging, 1992; 13:401-4). However, chronically elevating blood glucose concentrations (hyperglycemia) is detrimental and this is not a safe means to improve memory performance.

Brain Metabolism. The brain has a very high metabolic rate. For example, it uses 20 percent of the total oxygen consumed in a resting state. Large amounts of ATP are required by neurons of the brain for general cellular functions, maintenance of an electrical potential, synthesis of neurotransmitters and synaptic remodeling. Current models propose that under normal physiologic conditions, neurons of the adult human brain depend solely on glucose for energy. Since neurons lack glycogen stores, the brain depends on a continuous supply of glucose from the blood for proper function. Neurons are very specialized and can only efficiently metabolize a few substrates, such as glucose and ketone bodies. This limited metabolic capability makes brain neurons especially vulnerable to changes in energy substrates. Hence, sudden interruption of glucose delivery to the brain results in neuronal damage. Yet, if glucose levels drop gradually, such as during fasting, neurons will begin to metabolize ketone bodies instead of glucose and no neuronal damage will occur. Neuronal support cells, glial cells, are much more metabolically diverse and can metabolize many substrates; in particular, glial cells are able to utilize fatty acids for cellular respiration. Neurons of the brain cannot efficiently oxidize fatty acids and hence rely on other cells, such as liver cells and astrocytes to oxidize fatty acids and to produce ketone bodies. Ketone bodies are produced from the incomplete oxidation of fatty acids and are used to distribute energy throughout the body when glucose levels are low. In a normal Western diet, rich in carbohydrates, insulin levels are high and fatty acids are not utilized for fuel, hence blood ketone body levels are very low, and fat is stored and not used.

Current models propose that only during special states, such as neonatal development and periods of starvation, will the brain utilize ketone bodies for fuel. The partial oxidation of fatty acids gives rise to D-beta-hydroxybutyrate (D-3-β-hydroxybutyrate) and acetoacetate, which together with acetone, are collectively called ketone bodies. Neonatal mammals are dependent upon milk for development. The major carbon source in milk is fat (carbohydrates make up less then 12% of the caloric content of milk). The fatty acids in milk are oxidized to give rise to ketone bodies, which then diffuse into the blood to provide an energy source for development. Numerous studies have shown that the preferred substrates for respiration in the developing mammalian neonatal brain are ketone bodies. Consistent with this observation is the biochemical finding that astrocytes, oligodendrocytes and neurons all have capacity for efficient ketone body metabolism (for review, see Edmond, 1992). Yet only astrocytes are capable of efficient oxidation of fatty acids to ketone bodies.

The body normally produces small amounts of ketone bodies. However, because they are rapidly utilized, the concentration of ketone bodies in the blood is very low. Blood ketone body concentrations rise when on a low carbohydrate diet, during periods of fasting, and in diabetics. On a low carbohydrate diet, blood glucose levels are low, and pancreatic insulin secretion is not stimulated. This triggers the oxidation of fatty acids for use as a fuel source when glucose is limiting. Similarly, during fasting or starvation, liver glycogen stores are quickly depleted, and fat is mobilized in the form of ketone bodies. Since both a low carbohydrate diet and fasting do not result in a rapid drop of blood glucose levels, the body has time to increase blood ketone body levels. The rise in blood ketone bodies provides the brain with an alternative fuel source, and no cellular damage occurs. Since the brain has such high energy demands, the liver oxidizes large amounts of fatty acids until the body becomes literally saturated with ketone bodies. Therefore, when an insufficient source of ketone bodies is coupled with poor glucose utilization severe damage to neurons results. Since glial cells are able to utilize a large variety of substrates they are less susceptible to defects in glucose metabolism than are neurons. This is consistent with the observation that glial cells do not degenerate and die in AD (Mattson, 1998).

As discussed in the Metabolism and Alzheimer's Disease section, in AD, neurons of the brain are unable to utilize glucose and begin to starve to death. Since the defects are limited to the brain and peripheral glucose metabolism is normal, the body does not increase production of ketone bodies, therefore neurons of the brain slowly starve to death. Accordingly, there remains a need for an energy source for brain cells that exhibit compromised glucose metabolism. Compromised glucose metabolism is a hallmark of AD; hence administration of such an agent will prove beneficial to those suffering from AD.

Huntington's Disease

Huntington's Disease (HD) is a familial neurodegenerative disorder that afflicts 1 in 10,000 individuals. It is inherited in an autosomal dominant manner and is characterized by choreiform movements, dementia, and cognitive decline. The disease is produced by genes containing a variably increased (expanded) CAG repeat within the coding region. The size range of the repeats is similar in all diseases; unaffected individuals have fewer than 30 CAG repeats, whereas affected patients usually have more than 40 repeats. The disorder usually has a mid-life onset, between the ages of 30 to 50 years, but may in some cases begin very early or much later in life. The size of the inherited CAG repeat correlates with the severity and age of disease onset. The CAG triplet repeat produces a polyglutamine domain in the expressed proteins. The symptoms are progressive and death typically ensues 10 to 20 years after onset, most often as the result of secondary complications of the movement disorder.

The mutant gene produces huntingtin protein, whose function is unknown. The polyglutamine regions of Huntingtin interact with glyceraldehyde-3-phosphate dehydrogenase (GAPDH), a key glycolytic enzyme. While normal glutamine can bind with GAPDH and cause no harm to the enzyme, binding of mutant Huntingtin inhibits the enzyme. It is believed that the lack of energy being supplied to the brain cells, due to the interference of the Huntingtin protein with GAPDH, in part, causes neuron damage in the basal ganglia and the cerebral cortex. Mitochondrial dysfunction has also been implicated HD.

At least four other diseases are caused by the expanded CAG repeat, and thus also may implicate defective glucose metabolism. These include spinobulbar muscular atrophy, dentatorubral-pallidoluysian atrophy (DRPLA), spino-cerebellar ataxia type 1, and spino-cerebellar ataxia type 3.

Parkinson's Disease

Parkinson's Disease (PD) is widely considered to be the result of degradation of the pre-synaptic dopaminergic neurons in the brain, with a subsequent decrease in the amount of the neurotransmitter dopamine that is being released. Inadequate dopamine release, therefore, leads to the onset of voluntary muscle control disturbances symptomatic of PD.

The motor dysfunction symptoms of PD have been treated in the past using dopamine receptor agonists, monoamine oxidase binding inhibitors, tricyclic antidepressants, anticholinergics, and histamine H1-antagonists. Unfortunately, the main pathologic event, degeneration of the cells in substantia nigra, is not helped by such treatments. The disease continues to progress and, frequently after a certain length of time, dopamine replacement treatment will lose its effectiveness. In addition to motor dysfunction, however, PD is also characterized by neuropsychiatric disorders or symptoms. These include apathy-amotivation, depression, and dementia. PD patients with dementia have been reported to respond less well to standard L-dopa therapy. Moreover, these treatments have little or no benefit with respect to the neuropsychiatric symptoms. Impaired neuronal metabolism is believed to be a contributing factor to PD.

Epilepsy

Epilepsy, sometimes called a seizure disorder, is a chronic medical condition produced by temporary changes in the electrical function of the brain, causing seizures which affect awareness, movement, or sensation. There has been long experience with ketogenic diets, which mimic starvation, in children treated for epilepsy. The diet is a medical therapy and should be used under the careful supervision of a physician and/or dietician. The diet carefully controls caloric input and requires that the child eat only what has been included in the calculations to provide 90% of the day's calories as fats. However, such diets are generally unsuitable for use in adults due to: (1) adverse effects on the circulatory system from incorporation of long chain triglycerides as the primary fat in these diets into cholesterol and the effects of hyperlipidemia; (2) poor patient compliance due to the unappealing nature of the low carbohydrate diet.

Animal models of cognitive impairment greatly facilitate the study of such conditions including their physiology, neurology, anatomy, and pathology. Dogs provide a useful model as they demonstrate a pattern of age-associated cognitive decline in learning and memory, variable as to function of cognitive task (Adams B et al., 2000a; Chan A D F et al., 2002; Su M-Y et al., 1998; and, Tapp P D et al., 2003). The observed decline in dogs mirrors age-related cognitive declines seen in humans (Adams B et al. 2000b) and most likely is related to the same causes. Dogs also experience age-related reduction in regional cerebral metabolic rates for glucose (London E D et al., 1983). Dogs exhibit age-dependent changes in regional cerebral blood volume and blood-brain barrier permeability that may be related to changes in cognition, brain structure, and neuropathology with age (Tapp P D et al., 2005; and, Su M Y, 1998). Aged dogs develop neuropathology that is related to that seen in both successfully aging humans and patients with AD, such as beta amyloid protein (Cotman C W and Berchtold, 2002; and Cummings B J et al., 1996).

There is thus a need in the art to develop compositions and methods for the treatment and/or prevention of cognitive impairment, particularly in aging or geriatric mammals such as humans and companion animals.

Various publications, including patents, published applications, technical articles and scholarly articles are cited throughout the specification. Each of these cited publications is incorporated by reference herein, in its entirety. Full citations for publications not cited fully within the specification are set forth at the end of the specification.

### SUMMARY OF THE INVENTION

In one embodiment, the present invention includes a composition comprising medium chain triglycerides (MCTs), in an amount effective for improving performance in, or reversing, preventing, reducing, or delaying decline in one or more of cognitive function, memory, motor performance, cerebrovascular function, or behavior in an aging mammal, wherein said composition increases a circulating concentration of at least one type of ketone body in the mammal; and wherein the MCTs are of the formula:

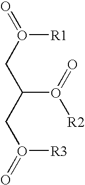

wherein the R1, R2, and R3 esterified to the glycerol backbone are each independently fatty acids having 5-12 carbons; wherein the aging mammal has reached at least about 50% of its life expectancy.

In another embodiment, the present invention includes a method for improving performance in, or reversing, preventing, reducing, or delaying decline in at least one of cognitive function memory, motor function, cerebrovascular function, or behavior in an aging mammal comprising the steps of: identifying an aging mammal having, or at risk of, decline in at least one of cognitive function memory, motor function, cerebrovascular function, or behavior; and administering to the mammal on an extended regular basis a composition comprising medium chain triglycerides (MCTs) as described previously in an amount effective to improve performance in, or to reverse, prevent, reduce, or delay decline in at least one of cognitive function memory, motor function, cerebrovascular function, or behavior in the mammal wherein said composition increases the circulating concentration of at least one type of ketone body in the mammal.

In another embodiment, the present invention includes a method for improving performance in, or reversing, preventing, reducing, or delaying decline in at least one of cognitive function, memory, motor function, cerebrovascular function, or behavior in an aging mammal comprising the steps of: (a) identifying an aging mammal not having an age-related cognitive impairment disease; and (b) administering to the mammal, on an extended regular basis, a composition comprising medium chain triglycerides (MCTs) as described previously in an amount effective to improve performance in, or reverse, prevent, reduce, or delay decline in at least one of cognitive function, memory, motor function, cerebrovascular function, or behavior in the mammal; (c) measuring the concentration of at least one type of ketone body, and at least one of cognitive function, memory, motor function, cerebrovascular function, or behavior in the mammal at least periodically for the duration of the administering step; (d) comparing at least one type of ketone body concentration and the measure of cognitive function, memory, motor function, cerebrovascular function, or behavior to that of a control mammal not receiving the administered composition; (e) correlating the ketone body concentration with the measure of cognitive function, memory, motor function, cerebrovascular function, or behavior thereby establishing the improvement in performance of, or reversal, prevention, reduction, or delay of the decline of at least one of cognitive function, motor function, cerebrovascular function, or behavior as a result of the administration of the composition.

The instant invention also includes a method for improving performance in, or reversing, preventing, reducing, or delaying decline in at least one of cognitive function, memory, motor function, cerebrovascular function, or behavior in a population of healthy aging mammals comprising the steps of: (a) identifying a population of healthy aging mammals not having age-related cognitive impairment; (b) dividing the population into at least a control group and one or more test groups; (c) formulating at least one diet-based delivery system for delivering a composition comprising medium chain triglycerides (MCTs) as previously described in an amount effective for elevating and maintaining an elevated level of at least one type of ketone body in the blood of an individual mammal; wherein, on an extended regular basis, each test group receives a formulation delivering a composition comprising MCTs and the control group does not receive any composition comprising MCTs; (d) comparing at least one of cognitive function, memory, motor function, cerebrovascular function, or behavior in the control and test groups; (e) determining which of the diet-based delivery systems for delivering the composition comprising MCTs was effective in improving performance in, or reversing, preventing, reducing, delaying decline of at least one of cognitive function, memory, motor function, cerebrovascular function, or behavior; and (f) administering a diet-based delivery system determined in step (e) to a population of aging mammals, thereby improving performance in, or reversing, preventing, reducing, delaying decline in at least one of cognitive function, memory, motor function, cerebrovascular function, or behavior.

### BRIEF DESCRIPTION OF THE DRAWINGS

FIG. 1 shows that treatment with MCTs led to a decline of 0.26 points at Day 90 in ADAS-Cog scores for the total ITT patient population, indicating that the MCT treated group showed a lessened decline in cognition as compared to the Placebo group.

FIG. 2 shows that treatment with MCTs led to a decline of 3.36 at Day 90 in ADAS-Cog scores for the subpopulation of ITT having the ApoE4(−) allele, indicating that the MCT treated subgroup having this allele showed an improvement in cognition over baseline and as compared to the Placebo group.

FIG. 3 shows that treatment with MCTs led to a decline throughout the course of the study of ADAS-Cog scores for the total ITT patient population, thus showing improved cognition relative to the Placebo.

FIG. 4 shows that treatment with MCTs led to a decline at Day 90 of ADCS-CGIS scores for the subpopulation of ITT having the ApoE4(−) allele, indicating that the MCT treated subgroup having this allele showed an improvement in cognition over the Placebo group.

FIG. 5 shows that treatment with MCTs led to a decline throughout the course of the study of ADCS-CGIS scores for the ITT population, indicating that the MCT treated subgroup having this allele showed an improvement in cognition over the Placebo group.

FIG. 6 shows that treatment with MCTs led to a decline throughout the course of the study of ADCS-CGIS scores for the subpopulation of ITT having the ApoE4(−) allele, indicating that the MCT treated subgroup having this allele showed an improvement in cognition over the Placebo group.

FIG. 7 shows a correlation between change in ADAS-Cog from Baseline to Day 90 to serum Cmax βHB levels for the MCT-treated ApoE4(−) subpopulation, indicating a significant pharmacologic response.

### DETAILED DESCRIPTION OF THE INVENTION

It is the novel insight of this invention that medium chain triglycerides (MCT) and their associated fatty acids are useful as a treatment and preventative measure in patients with any age-associated cognitive decline, such as Mild Cognitive Impairment, AAMI, or a dementing illness such as Alzheimer's disease, Huntington's disease, Parkinson's disease, and the like. These and other similar conditions are associated with reduced neuronal metabolism. As used herein, reduced neuronal metabolism refers to all possible mechanisms that could lead to a reduction in neuronal metabolism. Such mechanisms include, but are not limited to mitochondrial dysfunction, free radical attack, generation of reactive oxygen species (ROS), ROS-induced neuronal apoptosis, defective glucose transport or glycolysis, imbalance in membrane ionic potential, dysfunction in calcium flux, and the like.

MCT are composed of fatty acids with chain lengths of between 5-12 carbons. A diet rich in MCT results in high blood ketone levels. High blood ketone levels will provide an energy source for brain cells that have compromised glucose metabolism via the rapid oxidation of MCFA to ketone bodies, leading to improved performance in, and/or reversal, prevention, reduction, and/or delaying of decline in one or more of cognitive function, memory, motor performance, cerebrovascular function, and/or behavior. As used herein, “patient” refers to any mammal, including humans that may benefit from treatment of disease and conditions resulting from reduced neuronal metabolism.

### DEFINITIONS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be found in the specification and examples:

AD, Alzheimer's disease;

ALT, Alanine aminotransferase;

ANCOVA, analysis of covariance;

ANOVA, analysis of variance;

AVG, average;

BUN, blood urea nitrogen;

BW, body weight;

DNMP, delayed non-match to position;

F, female;

HDL, high-density lipoproteins;

LCT, long chain triglycerides

M, male;

MCFA, medium chain fatty acids

MCT, medium chain triglycerides;

MRI, magnetic resonance imaging;

SEM, standard error of the mean; and

VLDL, very low-density lipoproteins;

The metabolism of MCT differs from the more common long chain triglycerides (LCT) due to the physical properties of MCT and their corresponding medium chain fatty acids (MCFA). Due to the short chain length of MCFA, they have lower melting temperatures, for example the melting point of MCFA (C8:0) is 16.7° C., compared with 61.1° C. for the LCFA (C16:0). Hence, MCT and MCFA are liquid at room temperature. MCT are highly ionized at physiological pH, thus they have much greater solubility in aqueous solutions than LCT. The enhanced solubility and small size of MCT also increases the rate at which fine emulsion particles are formed. These small emulsion particles create increased surface area for action by gastrointestinal lipases. Additionally, medium chain 2-monoglycerides isomerize more rapidly than those of long chain length, allowing for more rapid hydrolysis. Some lipases in the pre-duodenum preferentially hydrolyze MCT to MCFA, which are then partly absorbed directly by stomach mucosa (Hamosh, M., Lingual and gastric lipases, Nutrition, 1990, 6:421-8). Those MCFA which are not absorbed in the stomach, are absorbed directly into the portal vein and not packaged into lipoproteins. LCFA are packaged in chylomicrons and transported via the lymph system, while MCFA are transported via the blood. Since blood transports much more rapidly than lymph, the liver is quickly perfused with MCFA.

In the liver the major metabolic fate of MCFA is oxidation. The fate of LCFA in the liver is dependent on the metabolic state of the organism. LCFA are transported into the mitochondria for oxidation using carnitine palmitoyltransferase I. When conditions favor fat storage, malonyl-CoA is produced as an intermediate in lipogenesis. Malonyl-CoA allosterically inhibits carnitine palmitoyltransferase I, and thereby inhibits LCFA transport into the mitochondria. This feedback mechanism prevents futile cycles of lipolysis and lipogenesis. MCFA are, to large extent, immune to the regulations that control the oxidation of LCFA. MCFA enter the mitochondria without the use of carnitine palmitoyltransferase I, therefore MCFA by-pass this regulatory step and are oxidized regardless of the metabolic state of the organism. Importantly, since MCFA enter the liver rapidly and are quickly oxidized, large amounts of ketone bodies are readily produced from MCFA. It is the novel insight of the inventor that MCTs may be administered outside of the context of a ketogenic diet. Therefore, in the present invention carbohydrates may be consumed at the same time as MCTs. This represents a significant advantage over the prior art, which only describes the use of MCTs in the context of a ketogenic diet. Such diets greatly restrict both carbohydrate and protein in the diet and are, in practice, extremely difficult with which to comply. The present invention represents a significant advantage over ketogenic diet prior art, in that in the present invention the subject is free follow any diet and does not have to adhere to any dietary restrictions.

The background of this invention supports the present invention in the following ways. (1) Neurons of the brain can use both glucose and ketone bodies for respiration. (2) The neurons of patients with any age-associated cognitive decline, such as Mild Cognitive Impairment, AAMI, or a dementing illness such as Alzheimer's disease, Huntington's disease, Parkinson's disease, and the like may have defects in glucose metabolism. (3) Aging may cause defects in metabolism that may underlie susceptibility to any age-associated cognitive decline, such as Mild Cognitive Impairment, AAMI, or a dementing illness such as Alzheimer's disease, Huntington's disease, Parkinson's disease, and the like. Hence, supplementation of patients with any age-associated cognitive decline, such as Mild Cognitive Impairment, AAMI, or a dementing illness such as Alzheimer's disease, Huntington's disease, Parkinson's disease, and the like with MCT will restore neuronal metabolism. As used herein, “high blood ketone levels” refers to levels of at least about 0.1 mM. More preferably, high blood ketone levels refers to levels in the range of 0.1 to 50 mM, more preferably in the range of 0.2-20 mM, more preferably in the range of 0.3-5 mM, and more preferably in the range of 0.5-2 mM.

“Medium chain triglycerides” or “MCTs” refers to any glycerol molecule ester-linked to three fatty acid molecules, each fatty acid molecule having 5-12 carbons. MCTs may be represented by the following general formula:

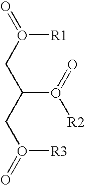

where R1, R2 and R3 are fatty acids having 5-12 carbons in the carbon backbone esterified to the a glycerol backbone. The structured lipids of this invention may be prepared by any process known in the art, such as direct esterification, rearrangement, fractionation, transesterification, or the like. For example, the lipids may be prepared by the rearrangement of a vegetable oil such as coconut oil. The length and distribution of the chain length may vary depending on the source oil. For example, MCTs containing 1-10% C6, 30-60% C8, 30-60% C10, 1-10% C10 are commonly derived from palm and coconut oils. MCTs containing greater than about 95% C8 at R1, R2 and R3 can be made by semi-synthetic esterification of octanoic acid to glycerin. Such MCTs behave similarly and are encompassed within the term MCTs as used herein.

“Effective amount” refers to an amount of a compound, material, or composition, as described herein that is effective to achieve a particular biological result. Such results include, but are not limited to, at least one of the following: enhancing cognitive function, improving memory, improving liver function, increasing daytime activity, improving learning, improving attention, improving social behavior, improving motor performance, and/or improving cerebrovascular function, particularly in aging or geriatric mammals. In various embodiments, “effective amount” refers to an amount suitable to reverse, reduce, prevent, or delay a decline in the above qualities, for example, cognitive function or performance, memory, learning rate or ability, problem solving ability, attention span and ability to focus on a task or problem, motor function or performance, social behavior, and the like. Preferably the reversal, prevention, reduction, or delay of a decline in an individual or population is relative to a cohort—e.g. a control mammal or a cohort population that has not received the treatment. Such effective activity may be achieved, for example, by administering the compositions of the present invention to a mammal or to a population of mammals.

The term “cognitive function” refers to the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness. “Enhanced cognitive function” or “improved cognitive function” refers to any improvement in the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness, as measured by any means suitable in the art.

“Behavior” is used herein in a broad sense, and refers to anything that a mammal does in response or reaction to a given stimulation or set of conditions. “Enhanced behavior” or “improved behavior” refers to any improvement in anything that a mammal does in response or reaction to a given stimulation or set of conditions.

“Motor performance” refers to the biological activity of the tissues that affect or produce movement in a mammal. Such tissues include without limitation muscles and motor neurons. “Enhanced motor performance” or “improved motor performance” refers to any improvement in the biological activity of the tissues that affect or produce movement in a mammal.

“Decline” of any of the foregoing categories or specific types of qualities or functions in an individual (characteristics or phenotypes) is generally the opposite of an improvement or enhancement in the quality or function. An “effective amount” (as discussed above) of a composition may be an amount required to prevent decline altogether or to substantially prevent decline (“prevent” decline), to reduce the extent or rate of decline (“reduce” decline), or delay the onset or progression of a decline (“delay” a decline), or lead to an improvement from a previous decline (“reversal of” or “reversing” a decline). Prevention, reduction, or delay of “decline” is frequently a more useful comparative basis when working with non-diseased aging mammals. Reversal, prevention, reduction, and delay can be considered relative to a control or cohort which does not receive the treatment, for example, the composition of interest. Reversal, prevention, reduction, or delay of either the onset of a detrimental quality or condition, or of the rate of decline in a particular function can be measured and considered on an individual basis, or in some embodiments on a population basis. The net effect of reversing, preventing, reducing, or delaying decline is to have less decrease in memory, cognitive, motor, or behavioral functioning per unit time, or at a given end point. In other words, ideally, for an individual or in a population, cognitive, motor, and behavioral functioning is maintained at the highest possible level for the longest possible time. For purposes herein, an individual can be compared to a control individual, group, or population. A population can likewise be compared to an actual individual, to normalized measurements for an individual, or to a group or population as is useful.

“Aging” as used herein means being of advanced age, such that the mammal has exceeded 50% of the average lifespan for its particular species. Aging mammals are sometimes referred to herein as “aged” or “geriatric” or “elderly.” Healthy aging mammals are those with no known diseases, particularly diseases relating to loss of cognitive impairment such as might confound the results. In studies using healthy aging mammals, cohort mammals are preferably also healthy aging mammals, although other healthy mammals with suitable memory, cognitive, motor, or behavioral functioning may be suitable for use as comparative specimens. If mammals with specific disease diagnoses, or memory, cognitive, motor, or behavioral limitations are used, then the cohort mammals should include mammals that are similarly diagnosed, or which present with similar indicia of the disease or memory, cognitive, motor, or behavioral limitation.

The present invention relates to any animal, preferably a mammal, and more preferably, humans. The compositions provided herein and below are generally intended for “long term” consumption, sometimes referred to herein as for ‘extended’ periods. “Long term” administration as used herein generally refers to periods in excess of one month. Periods of longer than two, three, or four months are preferred. Also preferred are more extended periods that include longer than 5, 6, 7, 8, 9, or 10 months. Periods in excess of 11 months or 1 year are also preferred. Longer terms use extending over 1, 2, 3 years or more are also contemplated herein. In the case of certain aging mammals, it is envisioned that the mammal would continue consuming the compositions for the remainder of its life on a regular basis. “Regular basis” as used herein refers to at least weekly, dosing with or consumption of the compositions. More frequent dosing or consumption, such as twice or thrice weekly are preferred. Still more preferred are regimens that comprise at least once daily consumption. The skilled artisan will appreciate that the blood level of ketone bodies, or a specific ketone body, achieved may be a valuable measure of dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

As used herein, the term “oral administration” or “orally administering” means that the mammal ingests, or a caretaker is directed to feed, or does feed, the mammal one or more of the compositions described herein. Wherein a human is directed to feed the composition, such direction may be that which instructs and/or informs the human that use of the composition may and/or will provide the referenced benefit, for example, enhancing cognitive function, improving memory, improving liver function, improving learning, improving attention, improving social behavior, improving motor performance, and/or improving cerebrovascular function, or preventing, reducing, or delaying a decline in such foregoing functions or qualities. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the composition (e.g., a label present on a container holding the composition).

The present invention provides a method of treating or preventing diseases of reduced neuronal metabolism, including any age-associated cognitive decline, such as Mild Cognitive Impairment, AAMI, or a dementing illness such as Alzheimer's disease, Huntington's disease, Parkinson's disease, and the like, comprising administering an effective amount of medium chain triglycerides to a patient in need thereof. Generally, an effective amount is an amount effective to either (1) reduce the symptoms of the disease sought to be treated or (2) induce a change relevant to treating the disease sought to be treated. For any age-associated cognitive decline, such as Mild Cognitive Impairment, AAMI, or a dementing illness such as Alzheimer's disease, Huntington's disease, Parkinson's disease, and the like, an effective amount includes an amount effective to: increase cognitive scores; improve memory; slow the progression of dementia; or increase the life expectancy of the affected patient. As used herein, medium chain triglycerides of this invention are represented by the following formula:

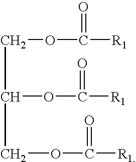

wherein R₁ is independently selected from the group consisting of a fatty acid residue esterified to a glycerol backbone having 5-12 carbons in the carbon backbone (C₅ to C₁₂ fatty acids), a saturated fatty acid residue esterified to a glycerol backbone having 5-12 carbons in the carbon backbone (C₅ to C₁₂ fatty acids), an unsaturated fatty acid residue esterified to a glycerol backbone having 5-12 carbons in the carbon backbone (C₅ to C₁₂ fatty acids), and derivatives of any of the foregoing. The structured lipids of this invention may be prepared by any process known in the art, such as direct esterification, rearrangement, fractionation, transesterification, or the like. For example the lipids may be prepared by the rearrangement of a vegetable oil such as coconut oil.

In a preferred embodiment, the method comprises the use of MCT wherein R₁ is a fatty acid containing a six-carbon backbone (tri-C6:0). Tri-C6:0 MCT are absorbed very rapidly by the gastrointestinal tract in a number of animal model systems. The high rate of absorption results in rapid perfusion of the liver, and a potent ketogenic response. In another preferred embodiment, the method comprises the use of MCT wherein R₁ is a fatty acid containing an eight-carbon backbone (tri-C8:0). In another preferred embodiment, the method comprises the use of MCT wherein R₁ is a fatty acid containing a ten-carbon backbone (tri-C10:0). In another preferred embodiment, the method comprises the use of MCT wherein R₁ is a mixture of C8:0 and C10:0 fatty acids. In another preferred embodiment, the method comprises the use of MCT wherein R₁ is a mixture of C6:0, C8:0, C10:0, and C12:0 fatty acids. Additionally, utilization of MCT can be increased by emulsification. Emulsification of lipids increases the surface area for action by lipases, resulting in more rapid hydrolysis and release of MCFA. Methods for emulsification of these triglycerides are well known to those skilled in the art.

In another preferred embodiment, the invention provides a method of treating or preventing diseases of reduced neuronal metabolism in patients with any age-associated cognitive decline, such as Mild Cognitive Impairment, AAMI, or a dementing illness such as Alzheimer's disease, Huntington's disease, Parkinson's disease, and the like, comprising administering an effective amount of free fatty acids, which may be derived from medium chain triglycerides, to a patient in need thereof.

In another preferred embodiment, the invention comprises the co-administration of emulsified MCT and L-carnitine or a derivative of L-carnitine. Slight increases in MCFA oxidation have been noted when MCT are combined with L-carnitine (Odle, J., New insights into the utilization of medium-chain triglycerides by the neonate: observations from a piglet model, J Nutr, 1997, 127:1061-7). Thus in the present invention emulsified MCT are combined with L-carnitine at doses required to increase the utilization of said MCT. The dosage of L-carnitine and MCT will vary according to the condition of the host, method of delivery, and other factors known to those skilled in the art, and will be of sufficient quantity to raise blood ketone levels to a degree required to treat and prevent AAMI or a dementing illness. Derivatives of L-carnitine which may be used in the present invention include but are not limited to decanoylcarnitine, hexanoylcarnitine, caproylcarnitine, lauroylcarnitine, octanoylcarnitine, stearoylcarnitine, myristoylcarnitine, acetyl-L-carnitine, O-Acetyl-L-carnitine, and palmitoyl-L-carnitine.

Therapeutically effective amounts of the therapeutic agents can be any amount or dose sufficient to bring about the desired anti-dementia effect and depend, in part, on the severity and stage of the condition, the size and condition of the patient, as well as other factors readily known to those skilled in the art. The dosages can be given as a single dose, or as several doses, for example, divided over the course of several weeks.

In one embodiment, the MCT or fatty acids are administered orally. In another embodiment, the MCT are administered intravenously. Oral administration of MCT and preparations of intravenous MCT solutions are well known to those skilled in the art.

Oral and intravenous administration of MCT or fatty acids result in hyperketonemia. Hyperketonemia results in ketone bodies being utilized for energy in the brain even in the presence of glucose. Additionally, hyperketonemia results in a substantial (39%) increase in cerebral blood flow (Hasselbalch, S. G., et al., Changes in cerebral blood flow and carbohydrate metabolism during acute hyperketonemia, Am J Physiol, 1996, 270:E746-51). Hyperketonemia has been reported to reduce cognitive dysfunction associated with systemic hypoglycemia in normal humans (Veneman, T., et al., Effect of hyperketonemia and hyperlacticacidemia on symptoms, cognitive dysfunction, and counterregulatory hormone responses during hypoglycemia in normal humans, Diabetes, 1994, 43:1311-7). Please note that systemic hypoglycemia is distinct from the local defects in glucose metabolism that occur in any age-associated cognitive decline, such as Mild Cognitive Impairment, AAMI, or a dementing illness such as Alzheimer's disease, Huntington's Disease, Parkinson's disease, and the like.

In another embodiment, the invention provides the subject compounds in the form of one or more prodrugs, which can be metabolically converted to the subject compounds by the recipient host. As used herein, a prodrug is a compound that exhibits pharmacological activity after undergoing a chemical transformation in the body. The said prodrugs will be administered in a dosage required to increase blood ketone bodies to a level required to treat and prevent the occurrence of any age-associated cognitive decline, such as Mild Cognitive Impairment, AAMI, or a dementing illness such as Alzheimer's disease, Huntington's Disease, Parkinson's disease, and the like. A wide variety of prodrug formulations are known in the art. For example, prodrug bonds may be hydrolyzable, such as esters or anhydrides, or enzymatically biodegradable, such as amides.

This invention also provides a therapeutic agent for the treatment or prevention of diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like, comprising medium chain triglycerides. In a preferred embodiment, the therapeutic agent is provided in administratively convenient formulations of the compositions including dosage units incorporated into a variety of containers. Dosages of the MCT are preferably administered in an effective amount, in order to produce ketone body concentrations sufficient to increase the cognitive ability of patients afflicted with diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like. For example, for the ketone body, D-beta-hydroxybutyrate, blood levels are raised to about 0.1-50 mM (measured by urinary excretion in the range of about 5 mg/dL to about 160 mg/dL), more preferably raised to about 0.2-20 mM, more preferably raised to about 0.3-5 mM, more preferably raised to about 0.5-2 mM, although variations will necessarily occur depending on the formulation and host, for example. Effective amount dosages of other MCT will be apparent to those skilled in the art. In one embodiment, an MCT dose will be in the range of 0.05 g/kg/day to 10 g/kg/day of MCT. More preferably, the dose will be in the range of 0.25 g/kg/day to 5 g/kg/day of MCT. More preferably, the dose will be in the range of 0.5 g/kg/day to 2 g/kg/day of MCT. Convenient unit dosage containers and/or formulations include tablets, capsules, lozenges, troches, hard candies, nutritional bars, nutritional drinks, metered sprays, creams, and suppositories, among others. The compositions may be combined with a pharmaceutically acceptable excipient such as gelatin, an oil, and/or other pharmaceutically active agent(s). For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof.

In a preferred embodiment, the subject is intravenously infused with MCT, MCFA (medium chain fatty acids) and/or ketone bodies directly, to a level required to treat and prevent the occurrence of diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like. Preparation of intravenous lipids, and ketone body solutions are well known to those skilled in the art.

In a preferred embodiment, the invention provides a formulation comprising a mixture of MCT and carnitine to provide elevated blood ketone levels. The nature of such formulations will depend on the duration and route of administration. Such formulations will be in the range of 0.05 g/kg/day to 10 g/kg/day of MCT and 0.05 mg/kg/day to 10 mg/kg/day of carnitine or its derivatives. In one embodiment, an MCT dose will be in the range of 0.05 g/kg/day to 10 g/kg/day of MCT. More preferably, the dose will be in the range of 0.25 g/kg/day to 5 g/kg/day of MCT. More preferably, the dose will be in the range of 0.5 g/kg/day to 2 g/kg/day of MCT. In some embodiments, a carnitine or carnitine derivative dose will be in the range of 0.05 g/kg/day to 10 g/kg/day. More preferably, the carnitine or carnitine derivative dose will be in the range of 0.1 g/kg/day to 5 g/kg/day. More preferably, the carnitine or carnitine derivative dose will be in the range of 0.5 g/kg/day to 1 g/kg/day. Variations will necessarily occur depending on the formulation and/or host, for example.

A particularly preferred formulation comprises a range of 1-500 g of emulsified MCT combined with 1-2000 mg of carnitine. An even more preferred formulation comprises 50 g MCT (95% triC8:0) emulsified with 50 g of mono- and di-glycerides combined with 500 mg of L-carnitine. Such a formulation is well tolerated and induces hyperketonemia for 3-4 hours in healthy human subjects.

In another embodiment, the invention provides the recipient with a therapeutic agent which enhances endogenous fatty acid metabolism by the recipient. The said therapeutic agent will be administered in a dosage required to increase blood ketone bodies to a level required to treat and prevent the occurrence of diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like. Ketone bodies are produced continuously by oxidation of fatty acids in tissues that are capable of such oxidation. The major organ for fatty acid oxidation is the liver. Under normal physiological conditions ketone bodies are rapidly utilized and cleared from the blood. Under some conditions, such as starvation or low carbohydrate diet, ketone bodies are produced in excess and accumulate in the blood stream. Compounds that mimic the effect of increasing oxidation of fatty acids will raise ketone body concentration to a level to provide an alternative energy source for neuronal cells with compromised metabolism. Since the efficacy of such compounds derives from their ability to increase fatty acid utilization and raise blood ketone body concentration they are dependent on the embodiments of the present invention.

Compounds that mimic the effect of increasing oxidation of fatty acids and will raise ketone body concentration include but are not limited to the ketone bodies, D-β-hydroxybutyrate and acetoacetate, and metabolic precursors of these. The term metabolic precursor, as used herein, refers to compounds that comprise 1,3 butane diol, acetoacetyl or D-β-hydroxybutyrate moieties such as acetoacetyl-1-1,3-butane diol, acetoacetyl-D-β-hydroxybutyrate, and acetoacetylglycerol. Esters of any such compounds with monohydric, dihydric or trihydric alcohols is also envisaged. Metabolic precursors also include polyesters of D-β-hydroxybutyrate, and acetoaoacetate esters of D-β-hydroxybutyrate. Polyesters of D-β-hydroxybutyrate include oligomers of this polymer designed to be readily digestible and/or metabolized by humans or mammals. These preferably are of 2 to 100 repeats long, typically 2 to 20 repeats long, and most conveniently from 3 to 10 repeats long. Examples of poly D-β-hydroxybutyrate or terminally oxidized poly-D-β-hydroxybutyrate esters useable as ketone body precursors are given below:

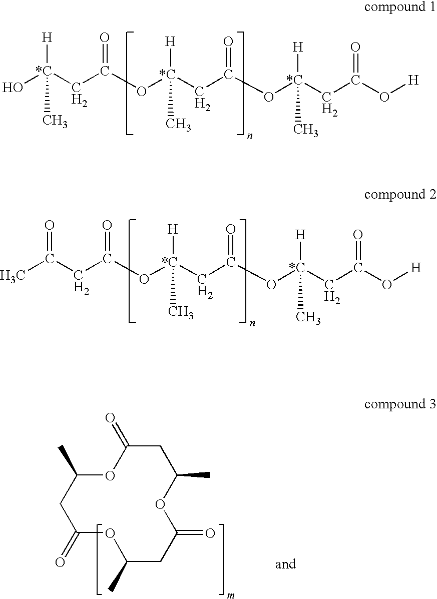

In each case, n is selected such that the polymer or oligomer is readily metabolized on administration to a human or mammal body to provide elevated ketone body levels in blood. Preferred values of n are integers of 0 to 1,000, more preferably 0 to 200, still more preferably 1 to 50, most preferably 1 to 20, particularly conveniently being from 3 to 5. In each case m is an integer of 1 or more, a complex thereof with one or more cations or a salt thereof for use in therapy or nutrition. Examples of cations and typical physiological salts are described herein, and additionally include sodium, potassium, magnesium, calcium, each balanced by a physiological counter-ion forming a salt complex, L-lysine, L-arginine, methyl glucamine, and others known to those skilled in the art. The preparation and use of such metabolic precursors is detailed in Veech, WO 98/41201, and Veech, WO 00/15216, each of which is incorporated by reference herein in its entirety.

The present invention provides a compound of the formula:

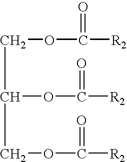

wherein R₂ is independently selected from the group consisting of R₁, an essential fatty acid esterified to a glycerol backbone, β-hydroxybutyrate esterified to a glycerol backbone, acetoacetate esterified to the glycerol backbone, compound 1 esterified to a glycerol backbone, compound 2 esterified to a glycerol backbone, and compound 3 esterified to a glycerol backbone, with the proviso that at least one of R₂ is R₁. This compound will provide increased levels of ketone bodies due to the MCT character of the molecule where R₂ is a ketone body precursor of the molecule. Additionally, where R₂ is an essential fatty acid, namely, linoleic or arachidonic acids, the compound has the additional advantage of providing the essential fatty acid.

Accordingly, the present invention also provides a method of treating or preventing diseases of reduced neuronal metabolism, in patients with any age-associated cognitive decline, such, as Mild Cognitive Impairment, AAMI, or a dementing illness such as, Alzheimer's disease, Huntington's disease, Parkinson's disease, and the like, comprising administering an effective amount of the foregoing compound to a patient in need thereof.

In another embodiment, the invention provides a therapeutic compound or mixture of compounds, the composition and dosage of which is influenced by the patients' genotype, in particular the alleles of the apolipoprotein E gene. In Example 3, for example, the inventor discloses that non-E4 carriers performed better than those with the E4 allele when elevated ketone body levels were induced with MCT. Also, those with the E4 allele had higher fasting ketone body levels and the levels continued to rise at the two hour time interval. Therefore, E4 carriers may require higher ketone levels or agents that increase the ability to use the ketone bodies that are present. Accordingly, a preferred embodiment consists of a dose of MCT combined with agents that increase the utilization of fats, MCT or ketone bodies. Examples of agents that increase utilization of fatty acids may be selected from a group comprising of, but not limited to, non-steroidal anti-inflammatory agents (NSAIDs), statin drugs (such as Lipitor® and Zocor®) and fibrates. Examples of NSAIDs include: aspirin, ibuprofen (Advil, Nuprin, and others), ketoprofen (Orudis KT, Actron), and naproxen (Aleve).

NSAIDs function, in part, as PPAR-gamma agonists. Increasing PPAR-gamma activity increases the expression of genes associated with fatty acid metabolism such as FATP (for review, see (Gelman, L., et al., An update on the mechanisms of action of the peroxisome proliferator-activated receptors (PPARs) and their roles in inflammation and cancer, Cell Mol Life Sci, 1999, 55:932-43)). Accordingly, a combination of MCT and PPAR-gamma agonists will prove beneficial to patients with decreased neuronal metabolism. In a preferred embodiment the PPAR-gamma agonist is an NSAID.

Statins are a class of drugs with pleiotropic effects, the best characterized being inhibition of the enzyme 3-hydroxy-3-methylglutaryl CoA reductase, a key rate step in cholesterol synthesis. Statins also have other physiologic affects such as vasodilatory, anti-thrombotic, antioxidant, anti-proliferative, anti-inflammatory and plaque stabilizing properties. Additionally, statins cause a reduction in circulating triglyceride rich lipoproteins by increasing the levels of lipoprotein lipase while also decreasing apolipoprotein C-III (an inhibitor of lipoprotein lipase) (Schoonjans, K., et al., 3-Hydroxy-3-methylglutaryl CoA reductase inhibitors reduce serum triglyceride levels through modulation of apolipoprotein C-III and lipoprotein lipase, FEBS Lett, 1999, 452:160-4). Accordingly, administration of statins results in increased fatty acid usage, which can act synergistically with MCT administration. This should prove especially beneficial to ApoE4 carriers. One embodiment of this invention would be combination therapy consisting of statins and MCT.

Fibrates, such as Bezafibrate, ciprofibrate, fenofibrate and Gemfibrozil, are a class of lipid lowering drugs. They act as PPAR-alpha agonists and similar to statins they increase lipoprotein lipase, apoAI and apoAII transcription and reduce levels of apoCIII (Staels, B., et al., Mechanism of action of fibrates on lipid and lipoprotein metabolism, Circulation, 1998, 98:2088-93). As such they have a major impact on levels of triglyceride rich lipoproteins in the plasma, presumably by increasing the use of fatty acids by peripheral tissues. Accordingly, the present invention discloses that fibrates alone or in combination with MCT would prove beneficial to patients with reduced neuronal metabolism such as those with any age-associated cognitive decline, such as Mild Cognitive Impairment, AAMI, or a dementing illness such as Alzheimer's disease, Huntington's disease, Parkinson's disease, and the like.

Caffeine and ephedra alkaloids are commonly used in over the counter dietary supplements. Ephedra alkaloids are commonly derived from plant sources such as ma-huang (Ephedra sinica). The combination of caffeine and ephedra stimulate the use of fat. Ephedra alkaloids are similar in structure to adrenaline and activate beta-adenergic receptors on cell surfaces. These adenergic receptors signal through cyclic AMP (cAMP) to increase the use of fatty acids. cAMP is normally degraded by phosphodiesterase activity. One of the functions of caffeine is to inhibit phosphodiesterase activity and thereby increase cAMP mediated signaling. Therefore caffeine potentiates the activity of the ephedra alkaloids. Accordingly, the present invention discloses that ephedra alkaloids alone can provide a treatment or prevention for conditions of reduced neuronal metabolism. Additionally, it is disclosed that ephedra alkaloids in combination with caffeine can provide a treatment or prevention for conditions of reduced neuronal metabolism. Accordingly, it is disclosed that a combination of MCT with ephedra, or MCT with caffeine, or MCT, ephedra alkaloids and caffeine together can provide a treatment or prevention for diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like.

Ketone bodies are used by neurons as a source of Acetyl-CoA. Acetyl-CoA is combined with oxaloacetate to form citrate in the Krebs' cycle, or citric acid cycle (TCA cycle). It is important for neurons to have a source of Acetyl-CoA as well as TCA cycle intermediates to maintain efficient energy metabolism. Yet, neurons lose TCA cycle intermediates to synthesis reactions, such as the formation of glutamate. Neurons also lack pyruvate carboxylase and malic enzyme so they cannot replenish TCA cycle intermediates from pyruvate (Hertz, L., et al., Neuronal-astrocytic and cytosolic-mitochondrial metabolite trafficking during brain activation, hyperammonemia and energy deprivation, Neurochem Int, 2000, 37:83-102). Accordingly, the present invention discloses that a combination of ketone bodies with a source of TCA cycle intermediates will be beneficial to conditions of reduced neuronal metabolism. TCA cycle intermediates are selected from a group consisting of citric acid, aconitic acid, isocitric acid, α-ketoglutaric acid, succinic acid, fumaric acid, malic acid, oxaloacetic acid, and mixtures thereof. One embodiment of the invention is a combination of TCA cycle intermediates with MCT in a formulation to increase efficiency of the TCA.

Another source of TCA cycle intermediates are compounds that are converted to TCA cycle intermediates within the body (TCA intermediate precursors). Examples of such compounds are 2-keto-4-hydroxypropanol, 2,4-dihydroxybutanol, 2-keto-4-hydroxybutanol, 2,4-dihydroxybutyric acid, 2-keto-4-hydroxybutyric acid, aspartates as well as mono- and di-alkyl oxaloacetates, pyruvate and glucose-6-phosphate. Accordingly, the present invention discloses that a combination of TCA intermediate precursors with ketone bodies will be beneficial for the treatment and prevention of diseases resulting from reduced metabolism. Also, the present invention discloses that MCT combined with TCA intermediate precursors will be beneficial for the treatment and prevention of diseases resulting from'reduced metabolism.

The present invention further discloses that additional sources of TCA cycle intermediates and Acetyl-CoA can be advantageously combined with ketone body therapy. Sources of TCA cycle intermediates and Acetyl-CoA include mono- and di-saccharides as well as triglycerides of various chain lengths and structures.

Further benefit can be derived from formulation of a pharmaceutical composition that includes metabolic adjuvants. Metabolic adjuvants include vitamins, minerals, antioxidants and other related compounds. Such compounds may be chosen from a list that includes but is not limited to; ascorbic acid, biotin, calcitriol, cobalamin, folic acid, niacin, pantothenic acid, pyridoxine, retinol, retinal (retinaldehyde), retinoic acid, riboflavin, thiamin, α-tocopherol, phytylmenaquinone, multiprenylmenaquinone, calcium, magnesium, sodium, aluminum, zinc, potassium, chromium, vanadium, selenium, phosphorous, manganese, iron, fluorine, copper, cobalt, molybdenum, iodine. Accordingly a combination of ingredients chosen from: metabolic adjuvants, compounds that increase ketone body levels, and TCA cycle intermediates, will prove beneficial for treatment and prevention of diseases of reduced neuronal metabolism, in patients with any age-associated cognitive decline, such as Mild Cognitive Impairment, AAMI, or a dementing illness such as Alzheimer's disease, Huntington's Disease, Parkinson's disease, and the like.

With regard to epilepsy, the prior art provides descriptions of ketogenic diets in which fat is high and carbohydrates are limited. In summary, the rationale of such diets is that intake of high amounts of fat, whether long-chain or medium-chain triglycerides, can increase blood ketone levels in the context of a highly-regimented diet in which carbohydrate levels are absent or limited. Limitation of carbohydrate and insulin are believed to prevent re-esterification in adipose tissue. In contrast to the prior art, the present invention provides for and claims the administration of medium chain triglycerides outside of the context of the ketogenic diet. Furthermore, the EXAMPLES section below provides exemplary formulations which include carbohydrates.

Although the ketogenic diet has been known for decades, there does not appear to be any prior art teaching or suggesting that MCT therapy be used to treat diseases of reduced neuronal metabolism, in patients with any age-associated cognitive decline, such as Mild Cognitive Impairment, AAMI, or a dementing illness such as Alzheimer's disease, Huntington's Disease, Parkinson's disease, and the like.

Additional metabolic adjuvants include energy enhancing compounds, such as Coenzyme CoQ-10, creatine, L-carnitine, n-acetyl-carnitine, L-carnitine derivatives, and mixtures thereof. These compounds enhance energy production by a variety of means. Carnitine will increase the metabolism of fatty acids. CoQ10 serves as an electron carrier during electron transport within the mitochondria. Accordingly, addition of such compounds with MCT will increase metabolic efficiency especially in individuals who may be nutritionally deprived.

Administration of MCT, and especially triglycerides composed of C6 and C8 fatty acid residues, result in elevated ketone body levels even if large amounts of carbohydrate are consumed at the same time (for overview see (Odle, J., New insights into the utilization of medium-chain triglycerides by the neonate: observations from a piglet model, J Nutr, 1997, 127:1061-7); see also copending United States Patent Provisional Patent Application Ser. No. 60/323,995, “Drug Targets for Alzheimer's Disease and Other Diseases Associated with Decreased Neuronal Metabolism,” filed Sep. 21, 2001). The advantages of the Applicant's approach are clear, since careful monitoring of what is eaten is not required and compliance is much simpler.

Further benefit can be derived from formulation of a pharmaceutical composition comprising MCT and other therapeutic agents which are used in the treatment of diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like. Such therapeutic agents include acetylcholinesterase inhibitors, acetylcholine synthesis modulators, acetylcholine storage modulators, acetylcholine release modulators, anti-inflammatory agents, estrogen or estrogen derivatives, insulin sensitizing agents, β-amyloid plaque removal agents (including vaccines), inhibitors of β-amyloid plaque formation, γ-secretase modulators, pyruvate dehydrogenase complex modulators, neurotrophic growth factors (e.g., BDNF), ceramides or ceramide analogs, and/or NMDA glutamate receptor antagonists (for overview of such treatments, see Selkoe 2001; Bullock 2002. While such treatments are still in the experimental stage it is the novel insight of the present invention that said treatments be advantageously combined with increased fatty acid/ketone body usage as described herein.

### ADVANTAGES

From the description above, a number of advantages of the invention for treating and preventing diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like, become evident:

(a) Current treatments for diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like, are merely palliative and do not address the reduced neuronal metabolism associated with these conditions. Ingestion of medium chain triglycerides as a nutritional supplement is a simple method to provide neuronal cells, in which glucose metabolism is compromised, with ketone bodies as a metabolic substrate.

(b) Increased blood levels of ketone bodies can be achieved by a composition or regimen rich in medium chain triglycerides.

(c) Medium chain triglycerides can be infused intravenously into patients or administered orally.

(d) Levels of ketone bodies can be easily measured in urine or blood by commercially available products (e.g., Ketostix®, Bayer, Inc.).

Accordingly, the reader will see that the use of medium chain triglycerides (MCT) or medium chain fatty acids as a treatment and preventative measure of diseases of reduced neuronal metabolism, in patients with any age-associated cognitive decline, such as Mild Cognitive Impairment, AAMI, or a dementing illness such as Alzheimer's disease, Huntington's disease, Parkinson's disease, and the like, provides a novel means of alleviating reduced neuronal metabolism associated with these conditions. It is the novel and significant insight of the present invention that use of MCT results in hyperketonemia which will provide increased neuronal metabolism for diseases of reduced neuronal metabolism in patients with any age-associated cognitive decline, such as Mild Cognitive Impairment, AAMI, or a dementing illness such as Alzheimer's disease, Huntington's disease, Parkinson's disease, and the like. Although the description above contains many specificities, these should not be construed as limiting the scope of the invention but merely as providing illustrations for some of the presently preferred embodiments of this invention. For example, supplementation with MCT may prove more effective when combined with insulin sensitizing agents such as vanadyl sulfate, chromium picolinate, and vitamin E. Such agents may function to increase glucose utilization in compromised neurons and work synergistically with hyperketonemia. In another example MCT can be combined with compounds that increase the rates of fatty acid utilization such as L-carnitine and its derivatives. Mixtures of such compounds may synergistically increase levels of circulating ketone bodies.

In one aspect, provided are compositions comprising medium chain triglycerides (MCT), in an amount effective for reversing, preventing, reducing, or delaying of one or more of cognitive function, memory, motor performance, cerebrovascular function, and/or behavior in an aging mammal. The aging or geriatric mammal will have reached at least about 50% of its life expectancy. The compositions increase circulating concentration of at least one ketone body in the mammal. The MCT are of the general formula [I]:

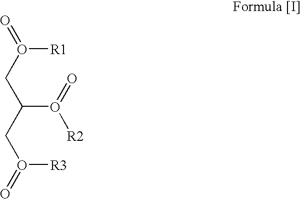

wherein the R1, R2, and R3 esterified to the glycerol backbone are each independently fatty acids having 5-12 carbons. In various embodiments, the compositions comprise MCT with greater than about 95% of the R1, R2, and R3 as C8 fatty acids. In one embodiment the remaining R1, R2, and R3 are preferably or even exclusively C6 or C10 fatty acids.

In other embodiments, the mammal is specifically a human. Other mammals within the scope of this invention are mammals such as companion animals, such as a pet or mammal in the care of a human for whether for a long term or briefly. In preferred embodiments, the companion mammal is a dog or cat.

In one embodiment, the mammal is a healthy aging mammal, as defined herein above. In such embodiments, the mammal will not be known to have overt signs or substantial symptoms or indicia of cognitive impairment, as determined by a skilled artisan. Although the mammal may have other health issues, even age-related health issues, they will be of such character as to not substantially impact the cognitive, motor, or behavioral functioning of the mammal. Thus, the skilled artisan will appreciate that it may be impossible to classify an aging or geriatric mammal as completely “healthy” —it is not necessary to do so to practice the methods and compositions provided herein. In other embodiments, the aging mammal is specifically understood to have age-related cognitive impairment, whether determined by formal diagnosis, or by its evidencing hallmarks of cognitive, memory, or motor impairments or behavioral indicia of such impairment or the like. In one embodiment, the mammal has a characteristic or phenotype associated with age-related cognitive impairment, for example the mammal has one or more of the following characteristic or phenotypic expressions of cognitive, motor, or behavioral difficulties associated with age. For example, decreased ability to recall, short-term memory loss, decreased learning rate, decreased capacity for learning, decreased problem solving skills, decreased attention span, decreased motor performance, increased confusion, or dementia, as compared to a control mammal not having the phenotype.

In one embodiment, the compositions of the invention are food compositions, such as pet foods. In certain embodiments, the composition is a food composition, further comprising in addition to the MCT, about 15-50% protein, 5-40% fat, 5-40% carbohydrate, each on a dry weight basis, and having a moisture content of 5-20%. In certain embodiments, the foods are intended to supply complete necessary dietary requirements. Also provided are compositions that are useful as snacks, nutrition bars, or other forms of food products or nutritional or dietary supplements, including tablets, capsules, gels, pastes, emulsions, caplets, and the like as discussed below. Optionally, the food compositions can be a dry composition, semi-moist composition, wet composition, or any mixture thereof.

In another embodiment, the compositions of the invention are food products formulated specifically for human consumption. These will include foods and nutrients intended to supply necessary dietary requirements of a human being as well as other human dietary supplements. In a one embodiment, the food products formulated for human consumption are complete and nutritionally balanced, while in others they are intended as nutritional supplements to be used in connection with a well-balanced or formulated diet.

In another embodiment, the composition is a food supplement, such as drinking water, beverage, liquid concentrate, gel, yoghurt, powder, granule, paste, suspension, chew, morsel, treat, snack, pellet, pill, capsule, tablet, or any other delivery form. The nutritional supplements can be specially formulated for consumption by a particular species or even an individual mammal, such as companion mammal, or a human. In one embodiment, the nutritional supplement can comprise a relatively concentrated dose of MCT such that the supplement can be administered to the mammal in small amounts, or can be diluted before administration to a mammal. In some embodiments, the nutritional supplement or other MCT-containing composition may require admixing with water or the like prior to administration to the mammal, for example to adjust the dose, to make it more palatable, or to allow for more frequent administration in smaller doses.

The MCT-containing compositions may be refrigerated or frozen. The MCT may be pre-blended with the other components of the composition to provide the beneficial amounts needed, may be emulsified, coated onto a pet food composition, nutritional or dietary supplement, or food product formulated for human consumption, or may be added to a composition prior to consuming it or offering it to a mammal, for example, using a powder or a mix.

In one embodiment, the compositions comprise MCT in an amount effective to enhance cognitive function and behavior in a mammal to which the composition has been administered. For pet foods and food products formulated for human consumption, the amount of MCT as a percentage of the composition is in the range of about 1% to about 50% of the composition on a dry matter basis, although a lesser or greater percentage can be supplied. In various embodiments, the amount is about 1.0%, 1.5%, 2.0%, 2.5%, 3.0%, 3.5%, 4.0%, 4.5%, 5.0%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%. 30%, 31%, 32%, 33%, 34%, 35%, 36%, 37%, 38%, 39%, 40%, 41%, 42%, 43%, 44%, 45%, 46%, 47%, 48%, 49%, 50% or more, of the composition on a dry weight basis. Nutritional supplements may be formulated to contain several fold higher concentrations of MCTs, to be amenable for administration to a mammal in the form of a tablet, capsule, liquid concentrated, or other similar dosage form, or to be diluted before administrations, such as by dilution in water, spraying or sprinkling onto a pet food, and other similar modes of administration. For a nutritional or dietary supplement, MCT alone may be administered directly to the mammal or applied directly to the mammal's regular food. Nutritional or dietary supplement formulations in various embodiments contain about 30% to about 100% MCTs, although lesser amounts may also used.

Sources of the MCT include any suitable source, semi-synthetic, synthetic or natural. Examples of natural sources of MCT include plant sources such as coconuts and coconut oil, palm kernels and palm kernel oils, and animal sources such as milk from any of a variety of species, e.g., goats.

In various embodiments, the compositions optionally comprise supplementary substances such as minerals, vitamins, salts, condiments, colorants, and preservatives. Non-limiting examples of supplementary minerals include calcium, phosphorous, potassium, sodium, iron, chloride, boron, copper, zinc, magnesium, manganese, iodine, selenium, and the like. Non-limiting examples of supplementary vitamins include vitamin A, any of the B vitamins, vitamin C, vitamin D, vitamin E, and vitamin K, including various salts, esters, or other derivatives of the foregoing. Additional dietary supplements may also be included, for example, any form of niacin, pantothenic acid, inulin, folic acid, biotin, amino acids, and the like, as well as salts and derivatives thereof. In addition, the compositions may comprise beneficial long chain polyunsaturated fatty acids such as the (n-3) and/or (n-6) fatty acids, arachidonic acid, eicosapentaenoic acid, docosapentaenoic acid, and docosahexaenoic acid, as well combinations thereof.

The compositions provided herein optionally comprise one or more supplementary substances that promote or sustain general neurologic health, or further enhance cognitive function. Such substances include, for example, choline, phosphatidylserine, alpha-lipoic acid, CoQ10, acetyl-L-carnitine, and herbal extracts such as Gingko biloba, Bacopa monniera, Convolvulus pluricaulis, and Leucojum aestivum.

In various embodiments, the pet food or dietary supplement compositions provided herein preferably comprise, on a dry weight basis, from about 15% to about 50% crude protein. The crude protein material comprise one or more proteins from any source whether animal, plant, or other. For example, vegetable proteins such as soybean, cottonseed, and peanut are suitable for use herein. Animal proteins such as casein, albumin, and meat protein, including pork, lamb, equine, poultry, fish, or mixtures thereof are useful.

The compositions may further comprise, on a dry weight basis, from about 5% to about 40% fat. The compositions may further comprise a source of carbohydrate. The compositions typically comprise from about 15% to about 40% carbohydrate, on a dry weight basis. Examples of such carbohydrates include grains or cereals such as rice, corn, sorghum, alfalfa, barley, soybeans, canola, oats, wheat, or mixtures thereof. The compositions also optionally comprise other components that comprise carbohydrates such as dried whey and other dairy products or by-products.

In certain embodiments, the compositions also comprise at least one fiber source. Any of a variety of soluble or insoluble fibers suitable for use in foods or feeds may be utilized, and such will be known to those of ordinary skill in the art. Presently preferred fiber sources include beet pulp (from sugar beet), gum arabic, gum talha, psyllium, rice bran, carob bean gum, citrus pulp, pectin, fructooligosaccharide additional to the short chain oligofructose, mannanoligofructose, soy fiber, arabinogalactan, galactooligosaccharide, arabinoxylan, or mixtures thereof. Alternatively, the fiber source can be a fermentable fiber. Fermentable fiber has previously been described to provide a benefit to the immune system of a companion animal. Fermentable fiber or other compositions known to those of skill in the art which provide a prebiotic composition to enhance the growth of probiotic microorganisms within the intestine may also be incorporated into the composition to aid in the enhancement of the benefit provided by the present invention to the immune system of an mammal. Additionally, probiotic microorganisms, such as Lactobacillus or Bifidobacterium species, for example, may be added to the composition. The skilled artisan will understand how to determine the appropriate amount of MCT to be added to a given composition. Such factors that may be taken into account include the type of composition (e.g., food composition, drink, dietary supplement, or food product formulated for human consumption), the average consumption of specific types of compositions by different mammals, the intended or required dose of MCT, the palatability and acceptability of the final product for the intended recipient or consumer, the manufacturing conditions under which the composition is prepared, the convenience for the purchaser, and packaging considerations. Preferably, the concentrations of MCT to be added to the composition are calculated on the basis of the energy and nutrient requirements of the mammal. The MCT can be added at any time during the manufacture and/or processing of the composition whether as part of a formulation of a pet food composition, dietary supplement, or food product for human consumption, or as a coating or additive to any of the foregoing.

The skilled artisan will appreciate that the compositions provided herein can be formulated and manufactured according to any suitable methods known in the art.

Another aspect of the invention provides methods for improving performance in or reversing, and/or preventing, reducing or delaying decline in one or more of cognitive function, memory, motor function and/or behavior in a mammal, particularly a geriatric mammal, particularly a human, comprising administering to the mammal a composition comprising MCT in an amount effective for improving performance in or reversing, and/or preventing, reducing or delaying decline in one or more of cognitive function, memory, motor function and/or behavior in the mammal

Thus in one aspect methods are provided for improving performance in or reversing, and/or preventing, reducing or delaying decline in one or more of cognitive function, memory, motor function and/or behavior in a mammal. In one embodiment the mammal is an aging or geriatric mammal. The methods comprise the steps of:

(a) identifying a mammal, such as an aging mammal, having, or at risk of, deficits or decline in at least one of cognitive function, memory, motor function, cerebrovascular function, or behavior; and

(b) administering to the mammal on an extended regular basis a composition comprising medium chain triglycerides (MCT) in an amount effective for improving performance in or reversing, and/or preventing, reducing or delaying decline in one or more of cognitive function, memory, motor function and/or behavior in the mammal. In certain embodiments, the composition increases the circulating concentration of at least one ketone body in the mammal. As with the compositions provided above, the MCT used herein are generally of the formula provided in Formula [I]:

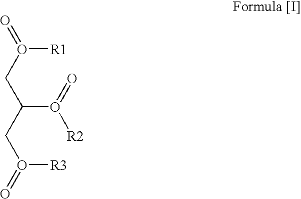

wherein the R1, R2, and R3 esterified to the glycerol backbone are each independently fatty acids having 5-12 carbons. In certain embodiments, greater than about 95% of the R1, R2, and R3 are 8 carbons in length. The remaining R1, R2, and R3 are 6-carbon or 10-carbon fatty acids in some embodiments.

In one embodiment, the method further comprises the step of monitoring the concentration of at least one ketone body in the mammal. The skilled artisan will appreciate that there are ways known to measure blood or plasma concentrations of ketone bodies collectively, or individually. All such methods are suitable for use herein in monitoring the ketone concentration in the mammal.

In one embodiment of the methods, the administered composition comprises MCT such that the amount of at least one of β-hydroxybutyrate, acetoacetate and acetone is raised in the blood of the mammal, particularly relative to a mammal not receiving the composition.

In various embodiments, the methods comprise an administration step wherein the composition comprises MCT in an amount effective for lowering the amount in the blood of the mammal of one or more of alanine, branched-chain amino acids, total lipoproteins, unsaturated fatty acids, or VLDL, or wherein the amount of each of alanine, branched-chain amino acids, total lipoproteins, unsaturated fatty acids, and VLDL is lowered in blood of the mammal.

In other embodiments, the composition administered comprises MCT in an amount effective for raising an amount in the blood of the mammal of one or more of glutamine, phenylalanine, HDL, or citrate, while in yet other embodiments, the amount of each of glutamine, phenylalanine, HDL, and citrate is raised in the blood of the mammal.

In one embodiment, the methods comprise an administration step wherein the composition comprises MCT in an amount effective for lowering the amount of each of alanine, branched-chain amino acids, total lipoproteins, unsaturated fatty acids, and VLDL, in addition to raising the amount of each of glutamine, phenylalanine, HDL, and citrate in the blood of the mammal.

In other embodiments of the methods, the administered composition comprises MCT in an amount effective for improving blood flow to the brain, or for improving the integrity of the blood brain barrier, or both. Such improvements can be measured with an individual over time, or relative to a control not receiving the composition.

In various embodiments provided herein the composition administered is a drink, food, nutritional or dietary supplement, or a drink or food product formulated for human consumption. In one embodiment, the mammal is a companion animal. In certain embodiments, the companion animal is a cat or dog.

The composition administered comprises at least about 1% to about 50% MCT on a dry weight basis in various applications of the methods. In certain embodiments, the administering step is on a regular basis comprising at least once daily. In some presently preferred embodiments the composition is administered as part of a daily regimen for at least about one week. A duration of two, three or even four weeks is also used. Administering the compositions for one to three months, or four months is exemplified herein. In other embodiments it is contemplated that administration will be extended for 4, 5, 6, 7, 8, 9, 10, 11 or even 12 months. In yet longer applications, administration periods extending 1, 2, 3, or more years are anticipated. In such embodiments, it may be useful to at least periodically monitor the mammal for ketoacidosis and the like, however, there is no evidence that the compositions or methods provided herein will result in ketoacidosis even after prolonged administration. In other embodiments the administration of the compositions is maintained for the remainder of the mammal's life (for example, the second half of the life expectancy for a mammal that has just recently attained aged or geriatric status as defined herein).

In one embodiment the composition is administered as part of a daily regimen for at least about one week, about three months, or about one year at a minimum.

In one embodiment the composition administered comprises MCT in an amount effective for lowering blood urea nitrogen or decreasing protein degradation. In another, the composition comprises MCT in an amount effective for lowering the amount or activity of alanine aminotransferase.

In one embodiment, the methods provided comprise an administration step wherein the composition comprises MCT in an amount effective for improving social behaviors of a mammal. Such improvement on the part of a companion animal can comprise interaction with its own or other species, such as a human.

For certain embodiments of this aspect the invention, the composition is a drink, food composition, nutritional or dietary supplement, or drink or food product formulated for human consumption as exemplified herein.

The compositions can be administered to the mammal by any of a variety of alternative routes of administration. Such routes include, without limitation, oral, intranasal, intravenous, intramuscular, intragastric, transpyloric, subcutaneous, rectal, and the like. Preferably, the compositions are administered orally.

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the mammal or otherwise contacted with or admixed with daily feed or food. When utilized as a daily feed or food, administration will be well known to those of ordinary skill.

Administration can also be carried out on a regular basis, for example, as part of a treatment regimen in the mammal. A treatment regimen may comprise causing the regular ingestion by the mammal of a composition comprising MCTs in an amount effective to enhance cognitive function, memory, and behavior in the mammal. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a regimen, administration can be multiple times per day. The goal of regular administration is to provide the mammal with the preferred daily dose of MCT, as exemplified herein.

The daily dose of MCT can be measured in terms of grams of MCT per kg of body weight (BW) of the mammal. The daily dose of MCT can range from about 0.01 g/kg to about 10.0 g/kg BW of the mammal. Preferably, the daily dose of MCT is from about 0.1 g/kg to about 5 g/kg BW of the mammal. More preferably, the daily dose of MCT is from about 0.5 g/kg to about 3 g/kg of the mammal. Still more preferably, the daily dose of MCT is from about 1 g/kg to about 2 g/kg of the mammal.

According to the methods of the invention, administration of the compositions comprising MCT, including administration as part of a treatment regimen, can span a period of time ranging from gestation through the entire life of the mammal. Preferably, the compositions comprising MCT are administered to geriatric mammals. Although different species of mammals reach advanced age at different rates, those of skill in the art will understand and appreciate when a given species has reached an advanced age. Determination of the appropriate age for a given mammal in which to administer compositions comprising. MCT can routinely be accomplished by those of skill in the art

In yet another of its several aspects, methods are provided for improving performance in or reversing, or preventing, reducing or delaying decline in one or more of cognitive function, memory, motor function and/or behavior in an aging mammal. The methods generally comprise the steps of:

(a) identifying an aging mammal not having an age-related cognitive impairment disease; (also sometimes referred to herein as a healthy aging mammal) and

(b) administering to the mammal, on an extended regular basis as defined herein, a composition comprising medium chain triglycerides (MCT) in an amount effective to improving performance in or reverse, or prevent, reduce, or delay decline in at least one of cognitive function, memory, motor function, cerebrovascular function, or behavior in the mammal;

wherein said composition increases the circulating concentration of at least one ketone body in the mammal;

(c) measuring the concentration of at least one ketone body, and at least one of cognitive function, memory, motor function, cerebrovascular function, or behavior in the mammal at least periodically for the duration of the administering step;

(d) comparing the at least one ketone body concentration and the measure of cognitive function, memory, motor function, cerebrovascular function, or behavior to that of a control mammal not receiving the administered composition;

(e) correlating the ketone body concentration with the measure of cognitive function, memory, motor function, cerebrovascular function, or behavior thereby establishing the prevention, reduction, or delay of the decline of at least one of cognitive function, motor function, cerebrovascular function, or behavior as a result of the administration of the composition.

In the methods provided in accordance with the foregoing and elsewhere herein the MCT are of the formula [I]:

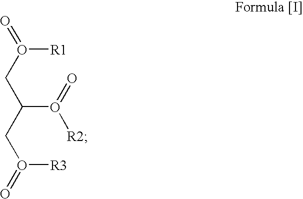

wherein the R1, R2, and R3 esterified to the glycerol backbone are each independently fatty acids having 5-12 carbons. In certain embodiments, greater than about 95% of the R1, R2, and R3 are 8 carbons in length. The remaining R1, R2, and R3 are 6-carbon or 10-carbon fatty acids in some embodiments.

In one embodiment of the methods, the administered composition comprises MCTs such that the amount of at least one of each of β-hydroxybutyrate, acetoacetate and acetone is raised in the blood of the mammal, particularly relative to a mammal not receiving the composition.

In various embodiments, the methods comprise an administration step wherein the composition comprises MCT in an amount effective for lowering the amount in the blood of the mammal of one or more of alanine, branched-chain amino acids, total lipoproteins, unsaturated fatty acids, or VLDL, or wherein the amount of each of alanine, branched-chain amino acids, total lipoproteins, unsaturated fatty acids, and VLDL is lowered in blood of the mammal.

In other embodiments, the composition administered comprises MCT in an amount effective for raising an amount in the blood of the mammal of one or more of glutamine, phenylalanine, HDL, or citrate, while in yet other embodiments, the amount of each of glutamine, phenylalanine, HDL, and citrate is raised in the blood of the mammal.

In one embodiment, the methods comprise an administration step wherein the composition comprises MCT in an amount effective for lowering the amount of each of alanine, branched-chain amino acids, total lipoproteins, unsaturated fatty acids, and VLDL, in addition to raising the amount of each of glutamine, phenylalanine, HDL, and citrate in the blood of the mammal.

In other embodiments of the methods, the administered composition comprises MCT in an amount effective for improving blood flow to the brain, or for improving the integrity of the blood brain barrier, or both. Such improvements in blood flow and integrity can be assessed over time in the mammal, or relative to a control not receiving the composition.

In various embodiments, the composition is a pet food, nutritional or dietary supplement, or a food product formulated for human consumption. The compositions, as well methods for its manufacture and administration as such are identical to that described in the previous aspect of the invention and such disclosure need not be repeated here in its entirety.

The composition administered comprises at least about 1% to about 50% MCTs on a dry weight basis in various applications of the methods. In certain embodiments, the administering step is on a regular basis comprising at least once daily. In some presently preferred embodiments the composition is administered as part of a daily treatment regimen for at least about one week. A duration of two, three or even four weeks is also used. Administering the compositions for one to three months, or four months is exemplified herein. In other embodiments it is contemplated that administration will be extended for 4, 5, 6, 7, 8, 9, 10, 11 or even 12 months. In yet longer applications, administration periods extending 1, 2, 3, or more years are anticipated. In such embodiments, it may be useful to at least periodically monitor the mammal for ketoacidosis and the like, however, there is no evidence that the compositions or methods provided herein will result in ketoacidosis even after prolonged administration. In other embodiments the administration of the compositions is maintained for the remainder of the mammal's life (for example, the second half of the life expectancy for a mammal that has just recently attained aged or geriatric status as defined herein).

In one embodiment the composition is administered as part of a daily treatment regimen for at least about one week, about three months, or about one year at a minimum.

In one embodiment the composition administered comprises MCT in an amount effective for lowering blood urea nitrogen or decreasing protein degradation. In another, the composition comprises MCTs in an amount effective for lowering the amount or activity of alanine aminotransferase.

In one embodiment, the methods provided comprise an administration step wherein the composition comprises MCT in an amount effective for improving social behaviors of a mammal including a companion animal. Such improvement can comprise interaction with its own or other species, such as a human.

In another aspect of the invention, provided are methods for improving performance in or reversing, or preventing, reducing or delaying decline in one or more of cognitive function, memory, motor function and/or behavior in a population of healthy aging mammals. Such methods are useful in the development and formulation of treatment regimens for improving performance in or preventing, reducing or delaying decline in cognitive, memory, motor, or behavioral function. The methods generally comprise:

(a) Identifying a population of healthy aging mammals. In preferred embodiments, the mammals do not have a diagnosis of any age-related cognitive impairment, nor obvious indicia of such conditions.

(b) Dividing the population into at least a control group and one or more test groups. The skilled artisan will appreciate that statistical methods of experimental design and available populations of mammals may dictate the number of groups into which the sample population can be properly divided.

(c) Formulating at least one delivery system or regimen for delivering a composition comprising medium chain triglycerides (MCT) in an amount effective for elevating and maintaining an elevated level of at least one ketone body in the blood of an individual mammal. The formulations of the treatment-based delivery system are based on the nutritional/dietary needs of the population including macro and micronutrients, energy requirements, and the like, and further comprise MCT as part of the diet. Preferably, the MCT are provided in the food formulation directly, but may also be included as a supplement thereto, in any form previously discussed herein with regard to other aspects of the invention. The MCT are of the formula [I] as provided above, and as previously wherein the R1, R2, and R3 esterified to the glycerol backbone are each independently fatty acids having 5-12 carbons.

A particular formulation is provided to each individual in the test group on an extended regular basis, as defined herein. Thus, each test group receives a formulation delivering a composition comprising MCTs, while the control group does not receive any composition comprising MCTs but rather receives a comparable formulation lacking MCTs but equivalent in terms of macro and micro nutrients, energy content, fiber, and the like.

(d) Comparing at least one of cognitive function, memory, motor function, cerebrovascular function, or behavior in the control and test groups. Art-known measures and methods can be readily applied here, and the skilled artisan can readily develop additional useful measures of such functions in accordance with the needs of the experiment or population.

(e) Determining which of the delivery systems for delivering the composition comprising MCT was effective in improving performance in or reversing, and/or preventing, reducing or delaying decline in one or more of cognitive function, memory, motor function and/or behavior.

(f) Finally, administering a treatment-based delivery system determined in step (e) above to a population of aging mammals, thereby reversing, and/or preventing, reducing or delaying one or more of cognitive function, memory, motor function and/or behavior.

In one embodiment, the “extended regular basis” for providing the test treatment comprises at least once daily for a period (duration) of at least about one week to about one year. Longer durations are contemplated for use herein, and such longer durations may involve fine-tuning prior iterations of formulated delivery systems to improve for example the prevention, reduction, or delay, or to improve palatability, convenience, or the like.

In one embodiment the methods further comprise the step of monitoring concentrations of at least one ketone body in each mammal in the control and test groups. In certain embodiments the amount at least one of β-hydroxybutyrate, acetoacetate or acetone is raised.

In various embodiments, the composition delivered by the system or regimen comprises MCT in an amount effective for lowering the blood level of one or more of alanine, branched-chain amino acids, total lipoproteins, unsaturated fatty acids, or VLDL. In another embodiment the level of each of alanine, branched-chain amino acids, total lipoproteins, unsaturated fatty acids, and VLDL is lowered. In another embodiment, the composition comprises MCT in an amount effective for raising the blood level of one or more of glutamine, phenylalanine, HDL, or citrate. In yet others, the level of each of glutamine, phenylalanine, HDL, and citrate is raised. In one embodiment wherein the composition comprises MCT in an amount effective for the lowering the level of each of alanine, branched-chain amino acids, total lipoproteins, unsaturated fatty acids, and VLDL, while the level of each of glutamine, phenylalanine, HDL, and citrate is raised.

In other embodiments of the methods, the administered composition comprises MCT in an amount effective for improving blood flow to the brain, or for improving the integrity of the blood brain barrier, or both. Such improvements in blood flow and integrity can be assessed over time in the mammal, or relative to a control not receiving the composition. They can also be relative, for example, to the control group on average.

In various embodiments, the composition is a drink, food, nutritional or dietary supplement, or a drink or food product formulated for human consumption. The compositions, as well methods for its manufacture and administration as such are identical to that described in the previous aspect of the invention and such disclosure provided there.

The composition administered comprises at least about 1% to about 50% MCTs on a dry weight basis in various applications of the methods. In certain embodiments, the administering step is on a regular basis comprising at least once daily. In some presently preferred embodiments the composition is administered as part of a daily treatment regimen for at least about one week. A duration of two, three or even four weeks is also used. Administering the compositions for one to three months, or four months is exemplified herein. In other embodiments it is contemplated that administration will be extended to 4, 5, 6, 7, 8, 9, 10, 11 or even 12 months. In yet longer applications, administration periods extending 1, 2, 3, or more years are anticipated. In such embodiments, it may be useful to at least periodically monitor the mammal for ketoacidosis and the like, however, there is no evidence that the compositions or methods provided herein will result in ketoacidosis even after prolonged administration. In other embodiments the administration of the compositions is maintained for the remainder of the mammal's life (for example, the second half of the life expectancy for a mammal that has just recently attained aged or geriatric status as defined herein).

In one embodiment the composition is administered as part of a daily treatment regimen for at least about one week, about three months, or about one year at a minimum.

In one embodiment the composition administered comprises MCT in an amount effective for lowering blood urea nitrogen or decreasing protein degradation. In another, the composition comprises MCT in an amount effective for lowering the amount or activity of alanine aminotransferase.

In one embodiment, the methods provided comprise an administration step wherein the composition comprises MCT in an amount effective for improving social behaviors of a companion animal. Such improvement can comprise interaction with its own or other species, such as a human.

Thus the scope of the invention should be determined by the appended claims and their legal equivalents, rather than by the examples given.

### EXAMPLES

The following examples are provided for illustrative purposes only and are not intended to limit the scope of the invention.

### Example 1

### Nutritional Drink

Nutritional drinks are prepared comprising the following ingredients: emulsified MCT in the range of 5 to 100 g/drink, L-carnitine in the range of 0.1 to 1 gram/drink, mix of vitamins and minerals at recommended daily levels, and a variety of flavorings.

### Example 2

### Additional formulations

Additional formulations can be in the form of Ready to Drink Beverages, Powdered Beverages, Nutritional Drinks, Food Bars, Puddings, other confections and the like. Formulations for such are clear to those skilled in the art.

A. Ready to Drink Beverage Ready to Drink Beverages are prepared so as to comprise the following ingredients: emulsified MCT in the range of 5-100 g/drink, L-carnitine in the range of 100-1000 mg/drink, and a variety of flavorings and other ingredients used to increased palatability, stability, etc.

B. Powdered Beverages MCT may be prepared in a dried form, useful for food bars and powdered beverage preparations. A powdered beverage may be prepared so as to comprise the following components per drink: dried emulsified MCT in the range of 10-50 g, L-carnitine in the range of 250-500 mg, sucrose in the range of 8-15 g, maltodextrin in the range of 1-5 g, flavorings 0-1 g and other ingredients used to increased palatability, stability, etc.

C. Food Bar A food bar would be comprised of: dried emulsified MCT 0.1-50 g, L-carnitine 250-500 mg, glycerin 1-5 g, corn syrup solids 5-25 g, cocoa 2-7 g, coating 15-25 g.

D. Gelatin Capsules Hard or soft gelatin capsules are prepared using the following ingredients: MCT 0.1-1000 mg/capsule, L-carnitine 250-500 mg/capsule, Starch, NF 0-600 mg/capsule; Starch flowable powder 0-600 mg/capsule; Silicone fluid 350 centistokes 0-20 mg/capsule. The ingredients are mixed, passed through a sieve, and filled into capsules.

E. Tablets Tablets are prepared so as to comprise the following ingredients: MCT 0.1-1000 mg/tablet; L-carnitine 250-500 mg/tablet; Microcrystalline cellulose 20-300 mg/tablet; Starch 0-50 mg/tablet; Magnesium stearate or stearate acid 0-15 mg/tablet; Silicon dioxide, fumed 0-400 mg/tablet; silicon dioxide, colloidal 0-1 mg/tablet, and lactose 0-100 mg/tablet. The ingredients are blended and compressed to form tablets.

F. Suspensions Suspensions are prepared so as to comprise the following ingredients: 0.1-1000 mg MCT; 250-500 mg L-carnitine; Sodium carboxymethyl cellulose 50-700 mg/5 ml; Sodium benzoate 0-10 mg/5 ml; Purified water 5 ml; and flavor and color agents as needed.

G. Parenteral Solutions A parenteral composition is prepared by stirring so as to comprise 1.5% by weight of MCT and L-carnitine in 10% by volume propylene glycol and water. The solution is made isotonic with sodium chloride and sterilized.

### Example 3

### Treating Alzheimer's Disease with Medium Chain Triglycerides

This example examined whether hyperketonemia improves cognition or memory in individuals with memory disorders. The goal of this trial was to test the hypothesis that elevation of serum beta-hydroxybutyrate (BHB) levels through a large oral dose of medium chain triglycerides will improve cognition, memory and attention performances in individuals with Alzheimer's disease (AD) or Mild Cognitive Impairment (MCI) as described below and in Reger, et al. (Neurobiology of Aging 25 (2004) 311-314).

Participants. The sample consisted of 20 individuals with memory disorders recruited from Western Washington. Potential subjects were excluded if they had diabetes mellitus, hypoglycemia, major psychiatric disorders, or other major medical or neurological disorders such as hypertension, hypotension, cardiac problems, or COPD. In addition, patients were excluded from the study if they were taking medications with CNS effects, such as anti-psychotics, anti-anxiolytics, and anti-hypertensives. However, subjects were allowed to participate if they were taking anti-depressants. Four participants were taking anti-depressants at the time of the study.

Table 1 describes the demographics of the sample. Fifteen subjects met NINCIDS/ADRDA criteria for probable AD. The remaining 5 subjects were diagnosed with Mild Cognitive Impairment, believed to be a prodromal phase of AD. Participants ranged in age from 61 to 84 years of age (mean=74.7), and 25% of the sample was female. The sample was well educated with an average of 13.3 years of education. Ninety percent of the sample was Caucasian. Two non-Caucasian subjects were identified as African-American and American Indian. Participants were typically in the mild to moderate stages of dementia. The mean baseline MMSE was 22.2. Forty-seven percent of the participants had at least one apoE ε4 allele.

TABLE 1
Sample Demographics and Medical Information

| Variable | Mean | SD |
|---|---|---|
| Age | 74.7 | 6.7 |
| Education | 13.3 | 3.25 |
| BMI | 26.0 | 3.7 |
| MMSE | 22.2 | 5.5 |
| | n | Sample % |
| AD | 15 | 75 |
| MCI | 5 | 25 |
| Female | 5 | 25 |
| E4+ | 10/19 | 53 |
| Non-Caucasian | 2 | 10 |

Note:
SD = Standard Deviation,
BMI = Body Mass Index,
MMSE = Mini-Mental State Examination,
E ε4+ = Subjects with at least one apoE ε4 allele

Procedures. Subjects were recruited through medical clinics, senior centers, and ads in newspapers. Prospective subjects' medical histories and cognitive complaints were telephone screened by research nurses. Individuals were then referred to the Memory Disorders Clinic at the VA Puget Sound Health Care System (VAPSHCS) for clinical and/or neuropsychological evaluation. Routine laboratory assays and EKGs were completed to assist in diagnosis and determination of research inclusion.

The study was conducted with a randomized, double-blind placebo controlled, crossover design. Initially, subjects were asked to come to the VAPSHCS for three visits. During each visit, subjects received one of two conditions in a randomized order: emulsified long chain triglycerides as a placebo (232 ml of heavy whipping cream) or medium chain triglycerides (MCT; 40 ml). NeoBee 895 (Stepan, Inc.) was used for MCT. MCT were blended with 152 ml of heavy whipping cream. Vanilla and non-caloric sweetener were added to the drink for taste.

Subjects arrived in the morning after a 12-hour fast and blood was drawn to determine BHB levels and apoE genotyping (first visit only). Subjects then consumed the blended test sample described above. About ninety minutes later, a second blood draw occurred and a 30-minute cognitive testing session ensued. A final blood draw was then completed. Study visits were conducted at least one week apart, and not more than four weeks apart.

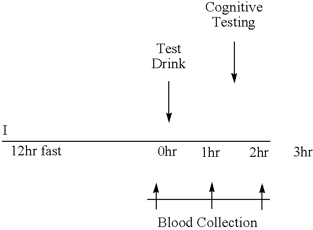

Neuropsychological Measures: Neuropsychological testing was performed by trained psychometrists using standardized procedures. A picture naming task, designed as a warm-up test, was completed at the beginning of the 30-minute test battery to reduce subject anxiety. The cognition and memory protocols included paragraph recall, the Stroop Color Word Interference Task, the Alzheimer's Disease Assessment Scale-Cognitive Subscale (ADAS-cog), and the Mini-Mental State Examination (MMSE). The Logical Memory subtest of the Wechsler Memory Scale-III was used as the model for the paragraph recall test. Subjects heard brief narratives containing 25 bits of information. They were asked to recall as much information as possible, both immediately after hearing the story and again after a 10 minute delay. This is a measure of short term memory in humans and is indicative of cognitive function. The Stroop Color Word Interference Task is a test of selective attention. The first two conditions require speeded reading of color words and speeded naming of colored blocks on a page. In the third condition, color names are printed in discordant ink colors and subjects are asked to state the color of the ink while inhibiting reading of the color words. Total reading time was recorded. The ADAS-cog is a mental status test designed specifically to rate the cognitive functioning of patients with Alzheimer's disease. Scores range from 1 to 70 with higher scores indicating increased impairment. The MMSE is a brief mental status test. Scores range from 0 to 30 with lower scores indicating increased impairment.

Beta-HydroxyButyrate Assays: Blood was processed immediately on the day of each subject's visit. Blood serum samples were kept in a −70° C. freezer until completion of the study. Beta-HydroxyButyrate (βHB, also referred to as BHB) levels were determined using a beta-hydroxybutyrate diagnostic kit (Sigma Diagnostics, Inc.). All samples were included in the assays and the lab was blinded to treatment conditions.

Results: treatment effects on βHB levels for βHB levels, a repeated measures ANCOVA was conducted with the ApoE genotype as the independent factor (ε4+ vs. 4−), and condition (treatment vs placebo) and time of blood draw (0, 90 min, and 120 min) as repeated factors and BMI as a covariate. βHB levels increased significantly with treatment (f[1, 15]=5.16, p<0.039), and there was a significant difference in βHB levels at different time points (f[2, 14]=5.22, p<0.01). Significant increases in βHB levels were observed 90-minutes after treatment (p=0.007). In addition, there was a significant interaction between e4 status and time of blood draw (f[2, 14]=3.76, p=0.036). Contrasts revealed that the βHB levels for ε4+ subjects continued to rise between the 90-minute and 120-minute blood draws in the treatment condition, while the βHB levels of ε4− subjects held constant (p<0.003). Table 2 lists the βHB means and standard deviations for each e4 group.

TABLE 2
Mean BHB Values by Treatment Condition and apoE ε4 Status

| | Baseline | | 90′ | | 120′ | |
|---|---|---|---|---|---|---|
| ε4 Status | Mean | SD | Mean | SD | Mean | SD |
| | PLACEBO | | | | | |
| ε4− | .04648 | .03565 | .07525 | .04780 | .09241 | .05803 |
| ε4+ | .14013 | .17946 | .15589 | .16760 | .18549 | .18405 |
| | MCT TREATMENT | | | | | |
| ε4− | .04150 | .02375 | .53784 | .31535 | .51515 | .25437 |
| ε4+ | .09504 | .08286 | .43022 | .18648 | .74142 | .37714 |

Note:
90′ = Values drawn 90 minutes after treatment;
120′ = Values drawn 120 minutes after treatment

Treatment Effects on Cognitive Performance and Memory. Repeated measures ANCOVAs were conducted with the apoE ε4 allele as the independent factor (£4+ vs. £4−) and condition (treatment vs placebo) as the repeated factor, BHB levels at the time of cognitive testing as a covariate, and cognitive measures as the dependent variables. For the ADAS-cog, subjects without the apoE ε4 allele showed improvement following MCT administration, whereas ε4+ subjects showed ADAS-cog Total Scores (lower scores indicate better performance) with slightly worse performance (table 2). This pattern resulted in a significant condition by £4 interaction (F[2, 14]=13.63, p=0.002).

The repeated measures ANCOVA with paragraph recall as the dependent measure revealed a trend interaction between the effects of treatment and BHB values measured just before testing (F[1, 14]=4.38, p=0.055). Subjects whose BHB levels were higher showed improved paragraph recall with MCT administration.

### Example 4

### Evaluation of oral MCT Administered for up to 90 Days in Subjects with Probable Alzheimer's Disease of Mild to Moderate Severity

In this example, a study was conducted to explore whether hyperketonemia improves cognitive functioning and memory in individuals with memory disorders, such as Alzheimer's disease. The goal of this trial was to test the hypothesis that sustained elevation of serum beta-hydroxybutyrate (βHB) levels through a large oral dose of medium chain triglycerides (MCT) will improve memory and attention performances in individuals with age associated cognitive decline or a dementing illness such as Alzheimer's disease or Mild Cognitive Impairment. The study was a randomized, double-blind, placebo-controlled, parallel, multi-center design. The subjects received either oral medium chain triglycerides (MCT) or placebo for ninety days followed by a two week washout period.

MCT or matching placebo was administered once a day for ninety days by mixing powder in one glass (approximately 8 oz.) of a liquid (i.e., water, juice, milk). For the first seven days of treatment, the subjects ingested 30 grams of powder (approximately 10 grams of Medium Chain Triglycerides) or placebo QD, increasing the dose to 60 gram QD (approximately 20 gram MCT) on Day 8 through Day 90. Following the end of the ninety day dosing period, subjects had a two week washout period.

Efficacy outcome measures were: a) Alzheimer's Disease Assessment Scale-Cognitive Subscale (ADAS-Cog), b) Alzheimer's Disease Cooperative Study-Clinician's Global Impression of Change (ADCS-CGIC) and c) Mini-Mental State. Exam (MMSE).

The Alzheimer's Disease Assessments Scale-Cognitive Subscale (ADAS-Cog) (Rosen et al. Am J Psychiatry 1984; 141(11): 1356-1364) is designed to measure cognitive symptom change in subjects with Alzheimer's disease. The standard 11 items are word-list recall, naming, commands, constructional praxis, ideational praxis, orientation, word recognition, spoken language ability, comprehension of spoken language, word-finding difficulty, and remembering test instructions.

The Alzheimer's Disease Cooperative Study-Clinician's Global Impression of Change (ADCS-CGIC) (Schneider et al. Alzheimer Disease and Associated Disorders 1997; 11(Suppl. 2) S22-S32) was used to assess change from the Baseline in the clinician's impression of change.

The Mini-Mental State Exam (MMSE) (Folstein et al. J. Psychia Res 1975; 12:189-198) was used as an assessment of mental status in five domains: orientation, registration, attention, recall and language.

Each subject was seen five times: at screening, at baseline, and at post baseline days 45, 90, and 104. At Visit 1 (screen), the following assessments were performed: demographics, medical/surgical history, NINCDS-ADRDA criteria, DSM-IV criteria, Modified Hachiniski Ischemia Scale, prior and concomitant medications, physical examinations, height, weight, vital signs, CT scan/MRI (performed if not previously done in last 18 months), ECG, TSH, B12, βHB serum level, safety laboratory assessments, ADAS-Cog, MMSE and Cornell Scale for Depression in Dementia.

Visit 2 (Baseline) occurred within 4 weeks (28 days) of Visit 1. The following assessments were conducted: adverse events (since initiation of Screen), concomitant medications, vital signs, ADAS-Cog, ADCS-CGIC and MMSE. Following completion of those assessments, eligible subjects were randomized, and the first dose (30 gm) of study medication was administered to the subject.

Visit 3 occurred 45 days (±3 days) after the Baseline visit. The following assessments were performed: adverse events, concomitant medications, vital signs, ADAS-Cog, ADCS-CGIC and MMSE. A blood sample was taken for serum βHB levels prior to dosing and 2 hr post-dosing.

Visit 4 occurred 90 days (±3 days) after the Baseline visit. The following assessments were performed: adverse events, concomitant medications, vital signs, ADAS-Cog, ADCS-CGIC, and MMSE. A blood sample was taken for serum βHB levels prior to dosing and 2 hr post-dosing.

Visit 5 occurred 104 days (±3 days) after the Baseline visit. The following assessments were performed: adverse events, concomitant medications, vital signs, weight, physical examination, ECG, safety labs, ADAS-Cog, ADCS-CGIC, and MMSE. A final blood sample was taken for serum βHB levels.

Change from Baseline at Day 90 was considered the primary measure of efficacy. Treatment comparisons for ADAS-Cog and MMSE (secondary outcome) were tested using ANCOVA with Treatment and Center as Factors and Age and Baseline scores as covariates. Treatment comparisons for ADCS-CGIC were done using Cochran-Mantel-Haenszel Tests. Treatment by genotype comparisons were done using a 2 way ANOVA with Treatment and ApoE4 status as variables. All comparisons used intent to treat populations (ITT) with last observation carried forward (LOCF).

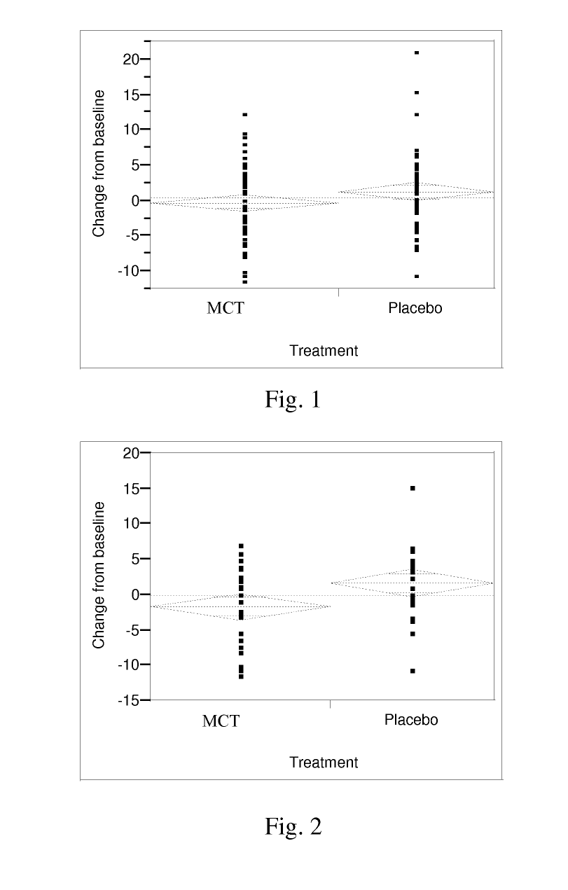

Results: ADAS-Cog. For all patients, when comparing MCTs and Placebo for change at Day 90 from Baseline, treatment with MCTs led to a decline of 0.26 points of total ADAS-Cog, whereas the Placebo group showed a 1.93 point decline, indicating that the MCT-treated patients showed lessened decline of cognitive function than the Placebo patients. See FIG. 1.

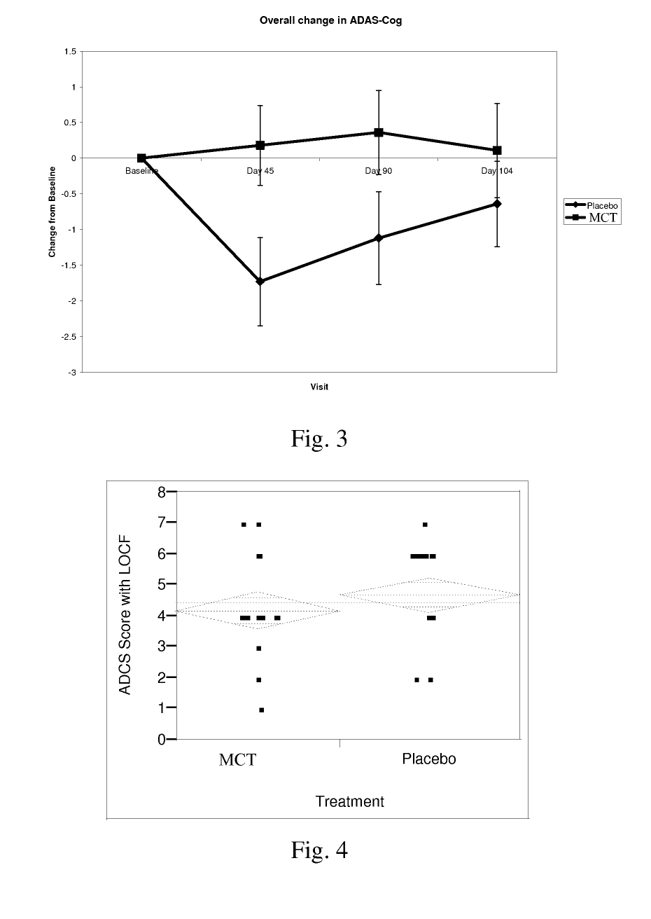

When comparing MCT and Placebo for change at Day 90 from Baseline for ApoE ε4(−) patients, the ApoE ε4(−) subjects improved cognitively (−1.75 points) on their ADAS-Cog scores, whereas the ApoE ε4(−) subjects on placebo declined (1.61 points) on their ADAS-Cog score. Scores on ADAS-Cog are inversely related to cognitive function. Therefore, lower scores represent improved performance on tests of memory, cognition, etc. The change in ADAS-Cog scores between MCT group and Placebo group was 3.36 points. See FIG. 2. Through the course of the study, subjects treated with MCTs generally showed improvement in cognition via their ADAS-Cog scores. See FIG. 3, (showing improvement in cognition from baseline data on the Y axis.)

AD Cooperative Study-Clinical Global Impression of Change (ADCS-CGIS). As for ADAS-Cog, lower scores indicate improved performance. After 90 days of treatment, ApoE ε4(−) subjects on MCTs scored an average of 4.17 points, whereas the ApoE ε4(−) subjects on Placebo scored an average of 4.68 points, showing decreased decline in the MCT patients. Therefore, improved scores were found in ApoE ε4(−) subjects treated with MCT. See FIG. 4.

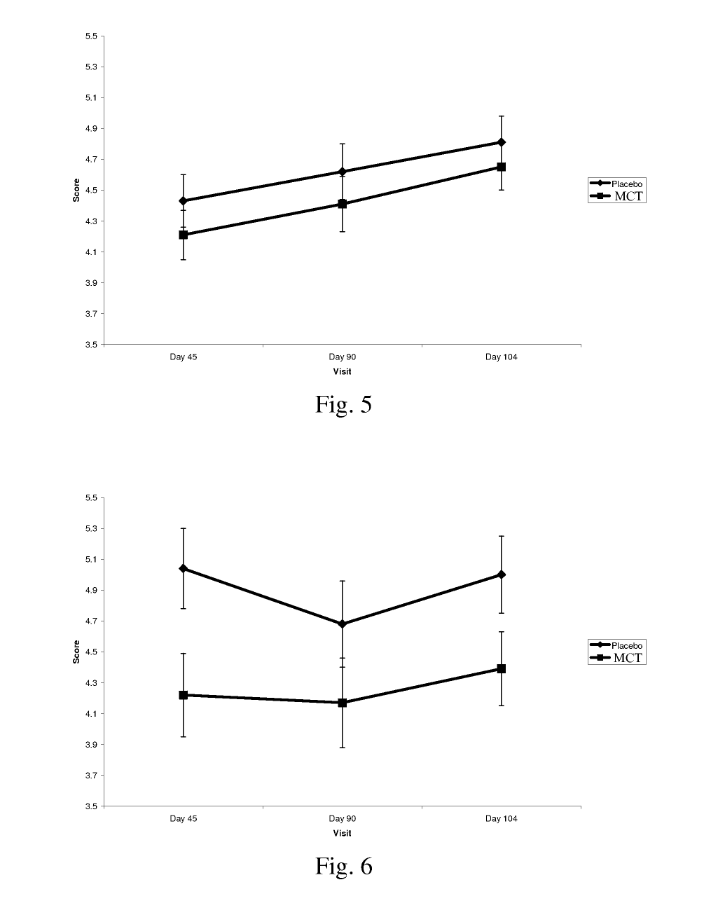

Through the course of the study, subjects treated with MCTs generally showed lowered scores on CGIC, indicating decrease in decline compared with Placebo, See FIG. 5. ApoE ε4(−) subjects showed lowered CGIC scores at Day 45 and Day 90. See FIG. 6.

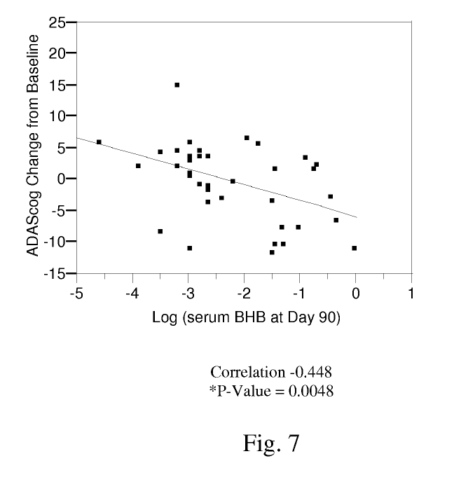

As discussed herein in the present Example, levels of β-hydroxybutyrate (βHB, a ketone body) were determined for patients in the study. It was found that there was a significant pharmacologic response between βHB plasma levels and ADAS-Cog scores in ApoE ε4(−) patients. FIG. 7 shows a correlation between change in ADAS-Cog from Baseline to Day 90 and serum Cmax βHB levels.

The results presented in this Example demonstrate that a formulation of medium chain triglycerides is able to improve cognitive impairment in an aged population in a statistically significant manner. The present Example shows that a daily, oral administration of MCTs improves performance, in particular on the ADAS-Cog scale, in an aged population in as few as 45 days. MCTs demonstrate even greater efficacy in the subset of the population lacking the apolipoprotein E epsilon 4 allele (also referred to herein as ApoE ε4− or ApoE4−).

MCTs are converted in the liver to ketone bodies, such as βHB, acetoacetate and acetone. Ketone bodies can be used as a metabolic substrate for a variety of cell types and as demonstrated herein in the present Example, the higher the level of serum ketone body βHB, the greater improvement seen in ADAS-Cog in ApoE ε4− subjects, strongly confirming the beneficial effects of daily MCT administration.

These results show that for Alzheimer's disease patients, particularly the subset of the patient population lacking the ApoE ε4 allele (making up almost 50% of the AD patient population), MCT treatment leads to improved cognition or memory in as little as 3 months.

### Example 5

### Evaluation of MCT as a Treatment for Age-Associated Memory Impairment (AAMI)

This example shows that MCT treatment has the potential to improve learning, memory or attention in subjects with Age-Associated Memory Impairment (AAMI). Criteria for AAMI include both subjective and objective evidence that memory loss has occurred since early adult life in the absence of disease or trauma of possible etiologic significance. AAMI is distinct from Alzheimer's disease (AD). People with AAMI are not at greater risk for developing AD (Youngjohn & Crook, 1993) and are appropriately described as having “normal” age-related memory loss.

Methods

Subjects

Subjects were a 67 year old male and a 63 year old female who met all diagnostic requirements for AAMI (Crook, T. H., 3rd, et al., Age-associated memory impairment: Proposed diagnostic criteria and measures of clinical change—Report of a National Institute of Mental Health work group, Dev Neruopsychol, 1986, 2:261-276). They were both physically healthy and showed no evidence of dementia according to standard diagnostic screening criteria (McKhann, G., et al., Clinical diagnosis of Alzheimer's disease: report of the NINCDS-ADRDA Work Group under the auspices of Department of Health and Human Serviced Task Force on Alzheimer's Disease, Neurology, 1984, 34:939-44). Indeed, both subjects obtained perfect scores on the most commonly used screening instrument for dementia, the Mini-Mental State

Examination (MMSE; (Folstein, M. F., et al., “Mini-mental state”. A practical method for grading the cognitive state of patients for the clinician, J Psychiatr Res, 1975, 12:189-98)). Both subjects did, however, score more than one standard deviation below the mean for young adults on a subscale of the Wechsler Memory Scale-Revised and complained of significant memory loss since early adult life (Crook, T. H., 3rd, et al., Assessment of memory complaint in age-associated memory impairment: the MAC-Q, Int Psychogeriatr, 1992, 4:165-76).

Study Design

This was a seven day open-label trial. Subjects given a battery of cognitive tests at baseline and then ingested 20 grams of MCT on each of the following seven mornings. The test substance (NeoBee 1053, (Stepan, Inc.) as the MCT source made up of approximately 50% C8 fatty acids and 50% C10 fatty acids randomly distributed on triglycerides) was mixed with a liquid (usually Boost™ high protein drink, (available from Novartis Medical Nutrition Fremont, Mich.)) and consumed as a breakfast drink. One hour after the final administration, subjects were retested on an alternate form of the same test battery.

Outcome Measures

A reliable and valid standardized, computerized test battery widely used in clinical trials (Larrabee, G. J. and Crook, T. H., 3rd, Estimated prevalence of age-associated memory impairment derived from standardized tests of memory function, Int Psychogeriatr, 1994, 6:95-104) was administered at baseline and at the conclusion of treatment. Changes in test scores over the seven day treatment period were calculated to assess treatment outcome. The specific tests are described as follows:

Name-Face Association Test (NFA)—Immediate and Delayed Recall

In this test, subjects are presented with a live video presentation of individuals introducing themselves by common first names. After a series of introductions, recall is assessed by showing the same individuals in a different order and asking the subject to provide the name of each person. There are two learning trials in which fourteen name-face pairs are presented and recall is assessed. Delayed recall is assessed thirty minutes later.

Facial Recognition Test—Delayed Non-Matching to Sample (DNM)

On the first trial of this test, subjects are presented with a single facial photograph on a touch screen monitor and asked to touch the face. On each of 24 subsequent trials, a new face is added to the array and the subject is required to identify the new face by touching it in the monitor. Each trial is separated from the preceding trial by an eight second interval during which the screen is black. Feedback is provided on each trial in the form of a red square that appears momentarily around the photograph if it is correctly identified

First-Last Names Test (FLN)—Immediate and Delayed Recall

In this test subjects are presented on the computer screen with six pairs of first and last names and asked to read each pair aloud. One pair is presented at a time. The last name corresponding in each pair is then presented and subjects are asked to provide the corresponding last name. This procedure is repeated three times with the same name pairs and then delayed recall is assessed after a 30 minute delay.

Telephone Dialing Test (TDT)

This task is a variation on the standard digit recall paradigm. Participants are presented with a series of ten-digit (long distance) telephone number on the monitor screen and asked to read them aloud. The number then disappears from the screen and subjects are instructed to dial the number on a representation of a touch-tone phone on the computer screen. On two of these trials the subject encounters “interference” in the form of a busy signal and must redial from memory. Four trials are conducted, with credit being given for each digit dialed in the correct position, regardless of errors made elsewhere in the sequence.

Results

Efficacy

As shown in Table 3A and 3B, both subjects improved on a number of tests administered.

TABLE 3A
Subject 1-67 year old male

| | Baseline | Post-Treatment | Percent |
|---|---|---|---|
| Test | Score | Score | Change |
| Name Face Association-Immediate | 11 | 15 | +36% |
| Recall (A + B) | | | |
| Name-Face Association-Delayed | 4 | 5 | +25% |
| Recall | | | |
| Facial Recognition | 19 | 21 | +11% |
| First-Last Names-Immediate | 3 | 4 | +25% |
| Recall (A + B) | | | |
| First-Last Names-Delayed Recall | 1 | 2 | +50% |
| Telephone Dialing-No Interference | 7.7 | 7.2 | −7% |
| Telephone Dialing-Interference | 5.4 | 5.3 | −2% |

TABLE 3B
63 year old female

| | Baseline | Post-Treatment | Percent |
|---|---|---|---|
| Test | Score | Score | Change |
| Name Face Association-Immediate | 13 | 17 | +31% |
| Recall (A + B) | | | |
| Name-Face Association-Delayed | 6 | 7 | +17% |
| Recall | | | |
| Facial Recognition | 17 | 19 | +12% |
| First-Last Names-Immediate | 5 | 5 | 0% |
| Recall (A + B) | | | |
| First-Last Names-Delayed Recall | 2 | 2 | 0% |
| Telephone Dialing-No Interference | 6.4 | 7.3 | +14% |
| Telephone Dialing-Interference | 5.3 | 5.8 | +9% |

Discussion

Clear improvements were seen on several of the tests, particularly the Name-Face Association Test, which is the test in the battery on which performance declines the greatest with advancing age. The magnitude of the change seen in this study shows MCT therapy is a promising treatment for AAMI, that is “normal” age-related memory loss.

### Example 6

### Changes in Blood Chemistry

Elevating serum ketone bodies in a mammal such as a human leads to changes in protein, lipid and carbohydrate metabolism. Such changes in blood chemistry are inherent upon treatment as described by the inventors in the present application and in the priority applications described and incorporated by reference herein. In this example, MCT is administered, to a human as described in Example 3, Example 4, and Example 5 and to a mammal (dog). Changes in blood chemistry in the human and the mammal are seen, including changes in serum lipid and protein concentrations. MCT administration is shown to induce ketosis even in the presence of abundant glucose. This hyperketonemia supplies cells of the body with an additional energy substrate in addition to the normal circulating glucose, lipid and protein energy reserves, preserving protein, lipid and glucose metabolism.

The following effects are observed in the human and in the mammal (dog) following administration of MCT as described in Example 3-5: 1) lowering of blood urea nitrogen, 2) decreasing of protein degradation, 3) lowering the amount or activity of alanine aminotransferase, 4) lowering the amount of alanine and branched-chain amino acids in the blood, and 5) raising the amount of glutamine and phenylalanine in the blood.

Positive alterations in lipid metabolism are evident. First, the human and mammal have lower amounts of total lipoproteins, unsaturated fatty acids, and Very Low Density Lipoproteins (VLDL), and higher amounts of High Density Lipoproteins (HDL).

Preservation of glucose metabolism is evident by elevation in blood citrate and glutamine levels.

It is observed that changes in carbohydrate, lipid and protein metabolism have other beneficial outcomes on the human and mammal (dog). In particular, the areas of motor performance, cerebrovascular function and changes in behavior result from the administration of MCT as described. It is noted that the blood flow to the brain and improving the integrity of the blood brain barrier occurs with MCT treatment as described. Additionally, the use of MCT leads to improvement in several social behaviors.

The present invention is not limited to the embodiments described and exemplified above, but is capable of variation and modification within the scope of the appended claims.

What is claimed is:

1. A composition comprising medium chain triglycerides (MCT), in an amount effective for improving performance in, or reversing, preventing, reducing, or delaying decline in one or more of cognitive function, memory, motor performance, cerebrovascular function, or behavior in an aging mammal, wherein said composition increases a circulating concentration of at least one type of ketone body in the mammal; and wherein the MCT are of the formula: 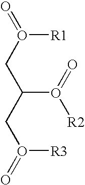
wherein the R1, R2, and R3 esterified to the glycerol backbone are each independently fatty acids having 5-12 carbons; wherein the aging mammal has reached at least about 50% of its life expectancy.

2. The composition of claim 1 wherein greater than about 95% of the R1, R2, and R3 are 8 carbons in length.

3. The composition of claim 2 wherein the remaining R1, R2, and R3 are 6-carbon or 10-carbon fatty acids.

4. The composition of claim 1, which is a food composition, further comprising on a dry weight basis about 5-50% protein, 5-40% fat, 5-40% carbohydrate, and having a moisture content of 5-20%.

5. The composition of claim 1, comprising at least about 1% to about 50% MCT on a dry weight basis.

6. The composition of claim 1, wherein the mammal is a human.

7. The composition of claim 6, wherein the human has a characteristic associated with an age-related cognitive impairment selected from the group of Age-Associated Memory Impairment, Mild Cognitive Impairment and Alzheimer's disease and related dementia.

8. The composition of claim 7 wherein the characteristic includes one or more of decreased ability to recall, short-term memory loss, decreased learning rate, decreased capacity for learning, decreased problem solving skills, decreased attention span, decreased motor performance, increased confusion, or dementia, as compared to a control mammal not having the characteristic.

9. A method for improving performance in, or reversing, preventing, reducing, or delaying decline in at least one of cognitive function memory, motor function, cerebrovascular function, or behavior in an aging mammal comprising the steps of:
identifying an aging mammal having, or at risk of, decline in at least one of cognitive function memory, motor function, cerebrovascular function, or behavior; and
administering to the mammal on an extended regular basis a composition comprising medium chain triglycerides (MCT) in an amount effective to improve performance in, or to reverse, prevent, reduce, or delay decline in at least one of cognitive function memory, motor function, cerebrovascular function, or behavior in the mammal wherein said composition increases the circulating concentration of at least one type of ketone body in the mammal; and wherein the MCT are of the formula: 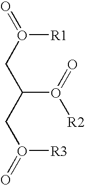
wherein the R1, R2, and R3 esterified to the glycerol backbone are each independently fatty acids having 5-12 carbons.

10. The method of claim 9 wherein greater than 95% of the R1, R2, and R3 are 8 carbons in length.

11. The method of claim 10 wherein the remaining R1, R2, and R3 are 6-carbon or 10-carbon fatty acids.

12. The method of claim 11 further comprising the step of monitoring the ketone body concentrations in the mammal.

13. The method of claim 9 wherein the amount of at least one of β-hydroxybutyrate, acetoacetate and acetone is raised in the blood of the mammal.

14. The method of claim 9 wherein the composition comprises MCT in an amount effective for lowering the amount in the blood of the mammal of one or more of alanine, branched-chain amino acids, total lipoproteins, unsaturated fatty acids, or VLDL.

15. The method of claim 14 wherein the amount of each of alanine, branched-chain amino acids, total lipoproteins, unsaturated fatty acids, and VLDL is lowered in blood of the mammal.

16. The method of claim 9 wherein the composition comprises MCT in an amount effective for raising an amount in the blood of the mammal of one or more of glutamine, phenylalanine, HDL, or citrate.

17. The method of claim 16 wherein the amount of each of glutamine, phenylalanine, HDL, and citrate is raised in the blood of the mammal.

18. The method of claim 17 wherein the composition comprises MCT in an amount effective for lowering the amount of each of alanine, branched-chain amino acids, total lipoproteins, unsaturated fatty acids, and VLDL.

19. The method of claim 9 wherein the composition comprises MCT in an amount effective for improving blood flow to the brain.

20. The method of claim 9 wherein the composition comprises MCT in an amount effective for improving the integrity of the blood brain barrier.

21. The method of claim 9, wherein the composition is a ready-to-drink beverage, powdered beverage formulation, nutritional or dietary supplement selected from the group consisting of gelatin capsule or tablet, suspension, parenteral solution, or a food product formulated for human consumption.

22. The method of claim 9, wherein the mammal is a human.

23. The method of claim 9, wherein the composition comprises at least about 1% to about 50 MCT on a dry weight basis.

24. The method of claim 9, wherein the administering step is on a regular basis comprising at least once daily.

25. The method of claim 24, wherein the composition is administered as part of a daily treatment regimen for at least about one week.

26. The method of claim 25, wherein the composition is administered as part of a daily treatment regimen for at least about three months.

27. The method of claim 9 wherein the composition comprises MCT in an amount effective for lowering blood urea nitrogen or decreasing protein degradation.

28. The method of claim 9 wherein the composition comprises MCT in an amount effective for lowering the amount or activity of alanine aminotransferase.

29. The method of claim 9 wherein the composition comprises MCT in an amount effective for lowering blood urea nitrogen or decreasing protein degradation.

30. A method for improving performance in, or reversing, preventing, reducing, or delaying decline in at least one of cognitive function, memory, motor function, cerebrovascular function, or behavior in an aging mammal comprising the steps of:
(a) identifying an aging mammal not having an age-related cognitive impairment disease; and
(b) administering to the mammal, on an extended regular basis, a composition comprising medium chain triglycerides (MCT) in an amount effective to improve performance in, or reverse, prevent, reduce, or delay decline in at least one of cognitive function, memory, motor function, cerebrovascular function, or behavior in the mammal;
wherein said composition increases the circulating concentration of at least one type of ketone body in the mammal; and wherein the MCT are of the formula: 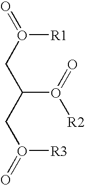
wherein the R1, R2, and R3 esterified to the glycerol backbone are each independently fatty acids having 5-12 carbons;
(c) measuring the concentration of at least one type of ketone body, and at least one of cognitive function, memory, motor function, cerebrovascular function, or behavior in the mammal at least periodically for the duration of the administering step;
(d) comparing at least one type of ketone body concentration and the measure of cognitive function, memory, motor function, cerebrovascular function, or behavior to that of a control mammal not receiving the administered composition;
(e) correlating the ketone body concentration with the measure of cognitive function, memory, motor function, cerebrovascular function, or behavior thereby establishing the improvement in performance of, or reversal, prevention, reduction, or delay of the decline of at least one of cognitive function, motor function, cerebrovascular function, or behavior as a result of the administration of the composition.

31. The method of claim 30, wherein greater than 95% of the R1, R2, and R3 are 8 carbons in length.

32. The method of claim 30, wherein the remaining R1, R2, and R3 are 6-carbon or 10-carbon fatty acids.

33. The method of claim 30, wherein the amount of at least one of β-hydroxybutyrate, acetoacetate or acetone is raised in the blood of the mammal.

34. The method of claim 30, wherein the composition comprises MCT in an amount effective for lowering, in the blood of the mammal, an amount of one or more of alanine, branched-chain amino acids, total lipoproteins, unsaturated fatty acids, or VLDL.

35. The method of claim 34, wherein the amount of each of alanine, branched-chain amino acids, total lipoproteins, unsaturated fatty acids, and VLDL is lowered.

36. The method of claim 30, wherein the composition comprises MCT in an amount effective for raising an amount in the blood of the mammal of one or more of glutamine, phenylalanine, HDL, or citrate.

37. The method of claim 36, wherein the amount of each of glutamine, phenylalanine, HDL, and citrate is raised.

38. The method of claim 30, wherein the composition comprises MCT in an amount effective for lowering the amount of each of alanine, branched-chain amino acids, total lipoproteins, unsaturated fatty acids, and VLDL.

39. The method of claim 30, wherein the composition comprises MCT in an amount effective for improving blood flow to the brain over time, or relative to the control mammal.

40. The method of claim 30, wherein the composition comprises MCT in an amount effective for improving the integrity of the blood brain barrier over time, or relative to the control mammal.

41. The method of claim 30, wherein the composition is a ready-to-drink beverage, powdered beverage formulation, nutritional or dietary supplement selected from the group consisting of gelatin capsule or table, suspension, parenteral solution, or a food product formulated for human consumption.

42. The method of claim 30, wherein the mammal is a human.

43. The method of claim 30, wherein the composition comprises at least about 1% to about 50% MCT on a dry weight basis.

44. The method of claim 30, wherein the administering step is on a regular basis comprising at least once daily.

45. The method of claim 44, wherein the composition is administered as part of a daily treatment regimen, for at least about one week to one year.

46. The method of claim 30 wherein the composition comprises MCT in an amount effective for lowering blood urea nitrogen or decreasing protein degradation.

47. The method of claim 30 wherein the composition comprises MCT in an amount effective for lowering the amount or activity of alanine aminotransferase.

48. The method of claim 30 wherein the composition comprises MCT in an amount effective for improving social behaviors.

49. A method for improving performance in, or reversing, preventing, reducing, or delaying decline in at least one of cognitive function, memory, motor function, cerebrovascular function, or behavior in a population of healthy aging mammals comprising the steps of:
(a) identifying a population of healthy aging mammals not having age-related cognitive impairment;
(b) dividing the population into at least a control group and one or more test groups;
(c) formulating at least one delivery system for delivering a composition comprising medium chain triglycerides (MCT) in an amount effective for elevating and maintaining an elevated level of at least one type of ketone body in the blood of an individual mammal; wherein the MCT are of the formula: 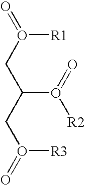
wherein the R1, R2, and R3 esterified to the glycerol backbone are each independently fatty acids having 5-12 carbons;
wherein, on an extended regular basis, each test group receives a formulation delivering a composition comprising MCT and the control group does not receive any composition comprising MCT;
(d) comparing at least one of cognitive function, memory, motor function, cerebrovascular function, or behavior in the control and test groups;
(e) determining which of the delivery systems for delivering the composition comprising MCT was effective in improving performance in, or reversing, preventing, reducing, delaying decline of at least one of cognitive function, memory, motor function, cerebrovascular function, or behavior; and
(f) administering a treatment-based delivery system determined in step (e) to a population of aging mammals, thereby improving performance in, or reversing, preventing, reducing, delaying decline in at least one of cognitive function, memory, motor function, cerebrovascular function, or behavior.

50. The method of claim 49 wherein the extended regular basis comprises at least daily for a period of at least about one week to about one year.

51. The method of claim 49 further comprising the step of monitoring the concentration of at least one type of ketone body in each mammal in the control and test groups.

52. The method of claim 49 wherein the amount of at least one of β-hydroxybutyrate, acetoacetate or acetone is raised.

53. The method of claim 49 wherein the composition comprises MCT in an amount effective for lowering the blood level of one or more of alanine, branched-chain amino acids, total lipoproteins, unsaturated fatty acids, or VLDL.

54. The method of claim 53 wherein the level of each of alanine, branched-chain amino acids, total lipoproteins, unsaturated fatty acids, and VLDL is lowered.

55. The method of claim 49 wherein the composition comprises MCT in an amount effective for raising the blood level of one or more of glutamine, phenylalanine, HDL, or citrate.

56. The method of claim 55 wherein the level of each of glutamine, phenylalanine, HDL, and citrate is raised.

57. The method of claim 56 wherein the composition comprises MCT in an amount effective for lowering the level of each of alanine, branched-chain amino acids, total lipoproteins, unsaturated fatty acids, and VLDL.

58. The method of claim 49 wherein the composition comprises MCT in an amount effective for improving blood flow to the brain.

59. The method of claim 49 wherein the composition comprises MCT in an amount effective for improving the integrity of the blood brain barrier.

60. The method of claim 49, wherein the mammal is a human.

61. The method of claim 49, wherein the composition comprises at least about 1% to about 50% MCTs on a dry weight basis.

62. The method of claim 49 wherein the composition comprises MCT in an amount effective for lowering blood urea nitrogen or decreasing protein degradation.

63. The method of claim 49 wherein the composition comprises MCT in an amount effective for lowering the amount or activity of alanine aminotransferase.

64. The method of claim 49 wherein the composition comprises MCT in an amount effective for improving social behaviors.
